(19) United States
(12) Patent Application Publication
(10) Pub. No.: US 2019/0366050 A1
(43) Pub. Date: Dec. 5, 2019

(54) CATHETER SECUREMENT SYSTEMS, KITS AND METHODS OF USING SAME

(71) Applicant: 3M INNOVATIVE PROPERTIES COMPANY, St. Paul, MN (US)

(72) Inventors: Jener De OLIVEIRA, São Paulo (BR); Steven B. HEINECKE, New Richmond, WI (US); Felipe S.R. BIZARRIA, São Paulo (BR); Adriana S.P. LOVON, São Paulo (BR); Elaine Cristina RAMIRES, São Paulo (BR)

(73) Assignee: 3M Innovative Properties Company, St. Paul, MN (US)

(21) Appl. No.: 16/477,952

(22) PCT Filed: Jan. 17, 2018
(86) PCT No.: PCT/IB2018/050284

§ 371 Date: Jul. 15, 2019

Related U.S. Application Data
(60) Provisional application No. 62/449,142, filed on Jan. 23, 2017.

(51) Int. Cl. A61M 25/02; A61F 13/02

(52) U.S. Cl. CPC A61M 25/02; A61F 2013/00412; A61F 13/0269

(57) ABSTRACT

Catheter securement systems, kits, and methods. The catheter securement systems include a base article that can be coupled to skin, and a coupling article having an anchor section and one or more legs configured to secure a catheter to the base article. The base article and the coupling article are configured to secure the catheter to the base article while allowing for removal and re-attachment of the catheter and the coupling article between two or more base articles attached to a patient while the base articles remain attached to the patient, or for repositioning of the catheter and coupling article on the same base article. Base articles can remain in position on the skin until it becomes necessary to change the base article or the catheter is removed from the patient. Catheter securement systems can be provided as a kit.

### FIELD

The present disclosure generally relates to catheter securement systems, kits, and methods of using the same, and particularly to systems configured to be secured to skin.

### BACKGROUND

During patient treatment (e.g., at hospitals, and particularly, in Intensive Care Units (ICUs)), the insertion of catheters and other tubes (all of which will be referred to herein collectively as “catheters”) can be required for different purposes, such as feeding, air supply, and/or liquid removal. In many instances, the catheters may need to be secured to the skin of a patient to keep the catheters from moving to, e.g., prevent them from being pulled out or otherwise moved in ways that may adversely affect their functioning.

One example of catheters that may be secured to the skin of a patient are urinary catheters (commonly referred to as Foley catheters), which are typically secured to a patient's thigh during use. The location at which the urinary catheters are attached is, however, changed to the opposite leg when the position of the patient is changed to prevent pressure ulcers, bed sores, etc. Repeated removal and re-attachment of the urinary catheters, however, can cause skin damage as adhesive articles are repeatedly attached and removed to the patient's thighs.

Many different devices have been developed to secure catheters to a patient's skin, see, e.g., the following published patent documents: U.S. Pat. No. 6,132,399 (Shultz); U.S. Pat. No. 8,251,957 (Kyvik, et al.); U.S. Pat. No. 9,248,259 (Kyvik, et al.); U.S. Pat. No. 9,463,303 (Nilson, et al.); China Patent App. No. 1751750 (Ma et al.); and China Patent App. No. 204502035 (Tang, et al.). Many of these devices are, however, complicated and/or too expensive, and/or not easily adaptable for different catheters (such as, e.g., urinary catheters that include a bifurcation at which a single tube separates into two tubes) to be widely used. As a result, many practitioners use different arrangements of adhesive tapes or do not secure the catheters at all. Further, the methods used to attach urinary catheters using adhesive tapes may vary between institutions and even between practitioners in the same institution.

### SUMMARY

As a result, there is a need for reliable repositionable catheter securement systems that can be used to securely and inexpensively attach catheters to patients that allow for repeated repositioning of the catheters at the same location and/or back and forth between at least two different locations without requiring adhesive articles to be removed and attached to the patient's skin whenever the catheter is repositioned to, e.g., increase the effectiveness of securement and patient comfort.

Securement systems as described herein allow for repositioning of a catheter at one location and/or between at least two locations on a patient (e.g., the patient's legs, etc.) when needed. In one or more embodiments, the catheter securement systems as described herein include a base article for securing the system to a selected location on a patient and a coupling article configured for attachment to the base article, while also being configured to secure a catheter that may, in one or more embodiments, include a bifurcated tube. The coupling article can be repositioned on the same base article and/or moved between two or more base articles attached to different locations on a patient without removing any base article from the skin or changing the position of any base article after attaching the base article to the patient. As a result, patient comfort may be enhanced and any potential risk for skin damage can be reduced.

In a first aspect, one or more embodiments of a catheter securement system as described herein include: a base article configured to be adhered to skin, the base article comprising a first major surface comprising a skin-contact adhesive and a second major surface opposite the first major surface and a coupling article. The coupling article includes: a carrier comprising a first major surface configured for repositionable attachment to the second major surface of the base article and a second major surface facing away from the first major surface; an anchor section attached to the second major surface of the carrier; and a capture section attached to the anchor section, the capture section comprising a first leg extending away from the anchor section, the first leg comprising a first end located distal from the anchor section.

In a second aspect, one or more embodiments of a catheter securement system as described herein include: a base article configured to be adhered to skin, the base article comprising a first major surface comprising a skin-contact adhesive and a second major surface opposite the first major surface and a coupling article configured for repositionable attachment to the second major surface of the base article. The coupling article includes an anchor section; a capture section attached to the anchor section, the capture section comprising: a first leg extending away from the anchor section, the first leg comprising a first end located distal from the anchor section; a second leg extending away from the anchor section, the second leg comprising a second end located distal from the anchor section; a bridge connecting the first leg to the second leg at a location distal from the anchor section, wherein the bridge is configured for repositionable attachment to the anchor section; and a capture aperture located between the anchor section, the bridge, the first leg, and the second leg.

In a third aspect, one or more embodiments of a catheter securement system as described herein include: a base article configured to be adhered to skin, the base article comprising a first major surface comprising a skin-contact adhesive and a second major surface opposite the first major surface and a coupling article comprising a first major surface configured for repositionable attachment to the second major surface of the base article. The coupling article includes; an anchor section and a capture section attached to the anchor section, the capture section comprising a first leg extending away from the anchor section, the first leg comprising a first end located distal from the anchor section.

In a fourth aspect, a kit comprising a package containing the catheter securement systems as described herein includes first and second base articles configured to be adhered to skin, the first and second base articles comprising a first major surface comprising a skin-contact adhesive and a second major surface opposite the first major surface.

In a fifth aspect, one or more embodiments of methods of securing a catheter as described herein include: adhesively attaching a first base article to a first location on a patient using skin-contact adhesive on a first major surface of the first base article; adhesively attaching a second base article to a second location on a patient using skin-contact adhesive on a first major surface of the second base article; securing a coupling article to a catheter; securing the catheter to the first base article using a coupling article;

removing the coupling article and the catheter from the first base article; and securing the catheter to the second base article using the coupling article after removing the coupling article and the catheter from the first base article.

In a sixth aspect, one or more embodiments of methods of securing a catheter as described herein include: adhesively attaching a first base article to a first location on a patient using skin-contact adhesive on a first major surface of the first base article; securing a coupling article to a catheter; securing the catheter to the first base article using a coupling article; removing the coupling article and the catheter from the first base article; and re-securing the catheter to the first base article using the coupling article after removing the coupling article and the catheter from the first base article.

In a seventh aspect, one or more embodiments of methods of securing a catheter as described herein include: adhesively attaching the base article of a catheter securement system as described herein to a first location on a patient using skin-contact adhesive on a first major surface of the base article; securing a coupling article of a catheter securement system as described herein to a catheter; and securing the catheter to the base article using the coupling article.

Unless specified or limited otherwise, the terms “attached,” “connected,” and “coupled,” and variations thereof, are used broadly and encompass both direct and indirect attachments, connections, and couplings.

The terms “layer,” “sheet,” and “dressing,” or variations thereof, are used to describe an article having a thickness that is small relative to its length and width.

The terms “polymer” and “polymeric material” refer to both materials prepared from one monomer such as a homopolymer or to materials prepared from two or more monomers such as a copolymer, terpolymer, or the like. Likewise, the term “polymerize” refers to the process of making a polymeric material that can be a homopolymer, copolymer, terpolymer, or the like. The terms “copolymer” and “copolymeric material” refer to a polymeric material prepared from at least two different monomers.

The term “repositionable” refers to the ability of an article or surface to be, at least initially, repeatedly coupled to (e.g., adhered to) and removed from a surface or substrate without substantial loss of coupling capability (e.g., adhesion) and without damage to either surface (e.g., article or underlying substrate) being coupled together. For example, a coupling article as described herein can be repositionable on a base article if the base article and the coupling article can be removed, or decoupled, from one another without causing damage to the base article or the coupling article. By way of example, some pressure-sensitive adhesives, mechanical fasteners, and non-tacky/cohesive polymeric materials are repositionable.

The phrase “mechanical fastener” generally refers to a fastener that includes two mating, or engagement, surfaces configured to be applied to one another, each mating surface having a plurality of engagement structures or features, such that engagement structures on one mating surface are configured to engage with the engagement structures on the opposing mating surface. In some embodiments, the mechanical fastener can include two flexible mating strips or layers. In some embodiments, the mechanical fastener can include a first mating surface comprising tiny, stiff protrusions shaped like hooks that are configured to engage a second mating surface comprising pliable loops (i.e., a “hook and loop fastener,” or “hook and pile fastener”). In some embodiments, the mechanical fastener can include inter-engaging hooks (e.g., self-engaging hooks) on both mating surfaces (i.e., a “hook and hook fastener” or a “self-engaging hook fastener”).

As used herein, the singular forms “a,” “an,” and “the” include plural referents unless the context clearly dictates otherwise. Thus, for example, reference to “a” or “the” component may include one or more of the components and equivalents thereof known to those skilled in the art. Further, the term “and/or” means one or all of the listed elements or a combination of any two or more of the listed elements.

Further, the term “comprises” and variations thereof do not have a limiting meaning where these terms appear in the accompanying description. Moreover, “a,” “an,” “the,” “at least one,” and “one or more” are used interchangeably herein.

Where used herein, the transitional phrases “consists of and “consisting of exclude any element, step, or component not specified. For example, “consists of or “consisting of used in a claim would limit the claim to the components, materials or steps specifically recited in the claim except for impurities ordinarily associated therewith (i.e., impurities within a given component). When the phrase “consists of or “consisting of appears in a clause of the body of a claim, rather than immediately following the preamble, the phrase “consists of or “consisting of limits only the elements (or components or steps) set forth in that clause; other elements (or components) are not excluded from the claim as a whole.

Where used herein, the transitional phrases “consists essentially of and “consisting essentially of are used to define systems, kits and methods that include materials, steps, features, components, or elements in addition to those literally disclosed, provided that these additional materials, steps, features, components, or elements do not materially affect the basic and novel characteristic(s) of the claimed invention. The term “consisting essentially of occupies a middle ground between “comprising” and “consisting of. Further, it should be understood that the herein-described systems, kits and methods may comprise, consist essentially of, or consist of any of the herein-described components and features, as shown in the figures with or without any additional feature(s) not shown in the figures. In other words, in some embodiments, the systems, kits and methods of the present invention may have any additional feature that is not specifically shown in the figures. In some embodiments, the systems, kits and methods of the present invention do not have any additional features other than those (i.e., some or all) shown in the figures, and such additional features, not shown in the figures, are specifically excluded from the systems, kits and methods.

The above summary is not intended to describe each embodiment or every implementation of the catheter securement systems, kits, or methods of using the same as described herein. Rather, a more complete understanding of the invention will become apparent and appreciated by reference to the following Detailed Description and claims in view of the accompanying figures of the drawing.

### BRIEF DESCRIPTION OF THE DRAWING

FIG. 1 is a plan view of one illustrative embodiment of a catheter securement system as described herein, the system shown in the form of a kit including two base articles and a coupling article.

FIG. 2 is an exploded edge view of one illustrative embodiment of one base article of the system of FIG. 1.

FIG. 3 is an exploded cross-sectional view of the coupling article of the system of FIG. 1 taken along line 3-3 in FIG. 1.

FIG. 4 is a cross-sectional view of the coupling article of the system of FIG. 1 taken along line 4-4 in FIG. 1.

FIG. 5 is a plan view of one illustrative embodiment of a base article as described herein with a bifurcated catheter positioned on the base article such that the bifurcation is positioned above the base article.

FIG. 6 is a plan view of the base article and bifurcated catheter of FIG. 5 after attachment of the anchor section of one illustrative embodiment of a coupling article as described herein to the base article and before attachment of the legs of the coupling article to the base article or the catheter.

FIG. 7 is a plan view of the base article, bifurcated catheter, and coupling article of FIG. 6 after attachment of the anchor section to the base article and attachment of the first leg of the coupling article (located to the left of the bifurcation as depicted in FIG. 7) to the base article, wherein the first leg extends over the supply tube of the catheter, wherein the second leg of the coupling article (located to the right of the bifurcation as depicted in FIG. 7) is not attached to the base article or the catheter.

FIG. 8 is a plan view of the base article, bifurcated catheter, and coupling article of FIG. 7 after attachment of the second leg of the coupling article (located to the right of the bifurcation as depicted in FIG. 7) to the base article, wherein the second leg extends between the first branch tube and the base article and over the second branch tube for attachment to the base article on an opposite side of the base article from the anchor section.

FIG. 9 is a plan view of another illustrative embodiment of a base article of a catheter securement system as described herein.

FIG. 10 is an exploded edge view of the base article FIG. 9.

FIG. 11 is a plan view of another illustrative embodiment of a coupling article of a catheter securement system as described herein.

FIG. 12 is an exploded edge view of the coupling article FIG. 11 FIG. 13 is a plan view of two base articles and coupling articles of FIG. 9-12 arranged on a kit sheet, with the coupling article pre-assembled on one of the base articles.

FIG. 14 is a plan view of another illustrative embodiment of a coupling article including a carrier and in use securing a catheter to a base article as described herein.

FIG. 15 is a cross-sectional view of the coupling article and base article of FIG. 14 taken along line 15-15 in FIG. 14.

FIG. 16 is an exploded edge view of another alternative illustrative embodiment of a coupling article of a catheter securement system as described herein.

FIGS. 17-19 are plan views of other illustrative embodiments of coupling articles of catheter securement systems as described herein.

FIG. 20A is a plan view of another illustrative embodiment of a coupling article that may be used in one or more embodiments of catheter securement systems as described herein.

FIG. 20B is a plan view of another illustrative embodiment of a coupling article that may be used in one or more embodiments of catheter securement systems as described herein.

FIG. 21 is a bottom perspective view of another illustrative embodiment of a coupling article for use in one or more embodiments of catheter securement systems as described herein.

FIG. 22 is an exploded cross-sectional view of the anchor section of the coupling article of FIG. 21 taken along line 22-22 in FIG. 21.

FIG. 23 is a top perspective view of the coupling article of FIG. 21.

FIG. 24 is a plan view of the coupling article of FIGS. 21-23 attached to one illustrative embodiment of a base article and securing one illustrative embodiment of a catheter to the base article.

FIG. 25 is a plan view of the opposite side of the coupling article and catheter of FIG. 24 after removal from the base article.

FIG. 26 is a top perspective view of another illustrative embodiment of a coupling article for use in one or more embodiments of catheter securement systems as described herein.

FIG. 27 is an exploded cross-sectional view of the anchor section of the coupling article of FIG. 26 taken along line 27-27 in FIG. 26.

FIG. 28 is a plan view of the coupling article of FIGS. 26-27 attached to one illustrative embodiment of a base article and securing one illustrative embodiment of a catheter to the base article.

### DETAILED DESCRIPTION

Catheter securement systems and methods of using the same are described herein. In one or more embodiments, the catheter securement systems described herein include a base article that can be coupled (i.e., adhered) to skin, and a coupling article having an anchor section and one or more legs configured to secure a catheter to the base article. The base article and the coupling article are configured to secure the catheter to the base article while allowing for removal and re-attachment of the catheter and the coupling article between two or more base articles attached to a patient while the base articles remain attached to the patient. The base articles can remain in position on the skin until it becomes necessary to change the base article or the catheter is removed from the patient.

The securement systems described herein can, in one or more embodiments, be provided as a kit, e.g., two or more base articles and a coupling article, all of which may, in one or more embodiments, be attached to a single release liner referred to herein as a kit sheet. The catheter securement systems described herein may enhance manufacturability, packaging, ease-of-use and standardization of application procedures or techniques.

In one or more embodiments, the base article can include a release agent (e.g., a release coating) on its back side to which a securing adhesive on the coupling article can be adhered to ensure that the coupling article (or at least a portion thereof) can be repositionable on the base article. Alternatively or additionally, in some embodiments, the base article can include a first mating surface of a mechanical fastener (e.g., hooks) on its back side to which a second mating surface of the mechanical fastener on the coupling article (e.g., loops) can be repositionably engaged.

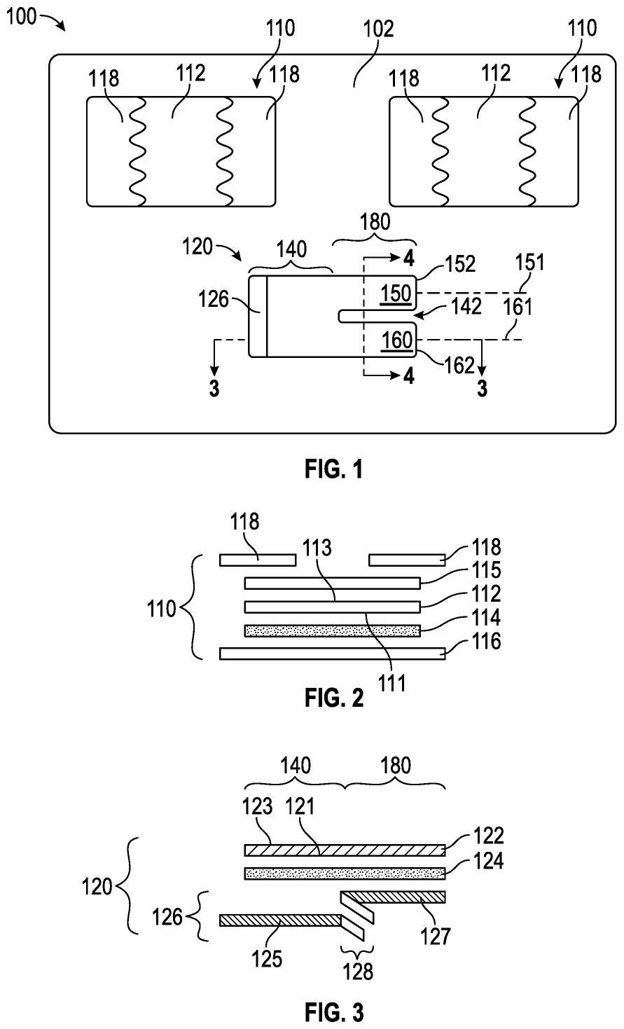

FIG. 1 depicts one illustrative embodiment of a catheter securement system 100 as described herein. By way of example only, the system 100 is shown as a kit including three elements of the system 100 all provided on a common sheet which may be referred to herein as a kit sheet 102. In one or more embodiments, the kit sheet 102 may be in the form of a release liner having at least one surface to which adhesive articles may be releasably secured. In one or more embodiments, the entire surface to which the adhesive articles are secured may function as a kit sheet, while in one or more alternative embodiments, the adhesive articles may be secured to subsections of the kit sheet 102 which are provided with release materials to allow for releasable attachment of the adhesive articles thereto. One or more embodiments of catheter securement system 100, when provided as a kit, may be located in a package for convenient delivery to a user. In one or more embodiments, the various components of the catheter securement system 100 in a package may be sterilized.

Although the catheter securement system 100 as depicted in FIG. 1 includes base articles 110 and coupling article 120 attached to a kit sheet 102, other alternative embodiments of catheter securement systems as described herein may include base articles 110 and coupling articles 120 as separate and discrete articles located in a package such that the base articles 110 and coupling articles 120 are not located on a common kit sheet 102.

As shown in FIG. 1, the depicted embodiment of catheter securement system 100 includes two base articles 110 and one coupling article 120 attached to kit sheet 102. In the depicted illustrative embodiment of system 100, each of the base articles 110 and the coupling article 120 provided in a sheet or dressing form that may, in one or more embodiments, include one or more layers of flexible materials or backings as described herein.

The base articles 110, one of which is shown in an exploded edge view in FIG. 2, can be configured (e.g., dimensioned, shaped, formed of appropriate materials, etc.) to be adhered to the skin of a patient at a selected location, e.g., on a leg, arm, torso, etc.

In the depicted illustrative embodiment as seen in FIG. 2, the base article 110 includes a backing layer 112 on which skin-contact adhesive 114 is located. In particular, the backing layer 112 provides a first (e.g., bottom) major surface 111 on which a skin-contact adhesive 114 is located, along with a second (e.g., top) major surface 113 opposite the first major surface 111. Although the backing layer 112 is depicted as being in the form of a single unitary layer, the backing layer 112 may, in one or more embodiments, include multiple layers and/or components as desired.

The base article 110 as depicted in FIG. 2 also includes a release liner 116 covering adhesive 114 until application of the base article 110 to a selected location on a patient is desired. Although the release liner 116 is depicted as a separate component in FIG. 2, the release liner 116 may, in one or more embodiments, be a portion of the kit sheet 102 as depicted in FIG. 1. In one or more alternative embodiments, the release liner 116 may be attached to the kit sheet 102 underneath the base article 110 to provide a release surface from which the skin-contact adhesive 124 on backing layer 112 can be removed.

The depicted illustrative embodiment of base article 110 depicted in FIG. 2 also includes tabs 118 on opposite ends of the base article. The tabs 118 may be useful in both removing the base article 110 from the release liner 116 (and/or kit sheet 102), as well as allowing a user to manipulate the base article 110 during placement on a patient. In one or more embodiments, the tabs 118 may be removable from the backing layer 112 after the base article 110 is in position on the skin of a patient at a selected location. Although tabs 118 on opposite ends of the base article 110 are used to facilitate delivery of the base article 110 in the depicted embodiment, base articles 110 used in one or more alternative embodiments of catheter securement systems described herein may use other structures and/or techniques to facilitate delivery of the base articles 110. For example, where the base articles 110 have a flexible backing layer 112, the tabs 118 may be replaced by a frame delivery system or any other suitable delivery system used in connection with the delivery of flexible backing layers to the skin of a patient.

In one or more embodiments, the base articles 110 each have a size that is at least as large or larger than the coupling article 120 when the coupling article is attached to the base articles and used to secure a catheter on the base articles 110 as described herein. In the depicted embodiment, each of the base articles 110 is provided in a generally rectangular form, although it should be understood that the base articles 110 may take any selected shape based on considerations such as, e.g., the intended location on a patient to which the base articles 110 are to be attached, the shape of the coupling articles 120 to be attached to the base articles 110, etc. In addition, although both base articles 110 depicted in FIG. 1 have the same size and shape, in one or more alternative embodiments, base articles 110 used in catheter securement systems described herein may have different shapes and/or sizes, both between different kits 100 as well as within the same kit 100.

As shown in the illustrative embodiment depicted in FIG. 1, one or more embodiments of the coupling articles 120 can have a shape that mimics the shape of the base articles 110, while also generally being smaller than the base article 110. Coupling articles 120 that match the shape of the base article 110 (e.g., being generally rectangular in the case of illustrative embodiments of base articles 110 and coupling articles 120) and that can be contained within the area of the base article 110 when the coupling article 120 is attached to the base article 110 can reduce the chances of attaching the coupling article 120 directly to the patient's skin outside of the boundaries of the base articles 110.

One or more embodiments of coupling articles of catheter securement systems described herein may have legs 150 and 160 that, like the illustrative embodiment of coupling article 120 that have both the same length and width, where the length of the legs 150 and 160 is measured from the anchor section 140 to the distal ends of the legs 150 and 160 while the width is measured transverse to the length of each leg. In one or more alternative embodiments, the legs of coupling articles may have different lengths and/or different widths.

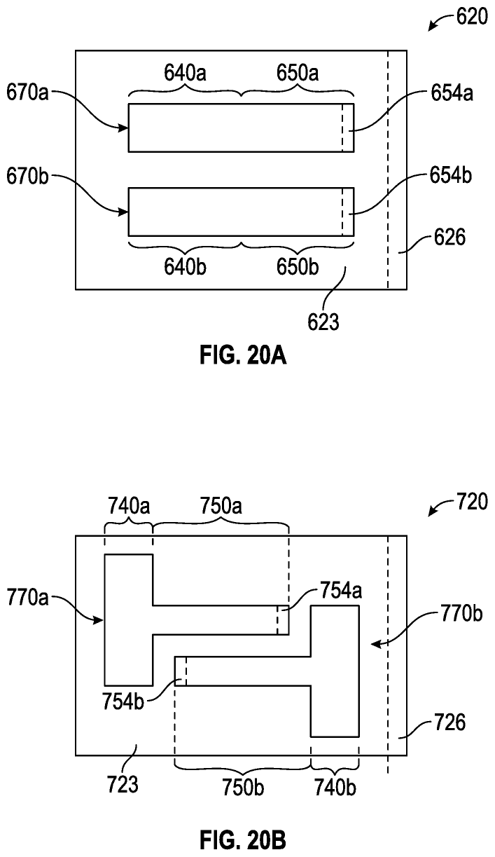

Although the depicted illustrative embodiment of coupling article 120 includes two legs 150 and 160 extending from an anchor section 140, one or more alternative embodiments of coupling articles that may be used in one or more embodiments of catheter securement systems as described herein may include three or more legs extending from an anchor section as needed to secure a catheter on a patient as described herein. Still other alternative embodiments of coupling articles as described herein may include only a single leg extending from an anchor section (see, e.g., FIGS. 19-20).

Further, although the kit 100 is depicted as including two base articles 110 and one coupling article 120, catheter securement systems as described herein when provided in a kit form may include three or more base articles 110 and/or two or more coupling articles 120.

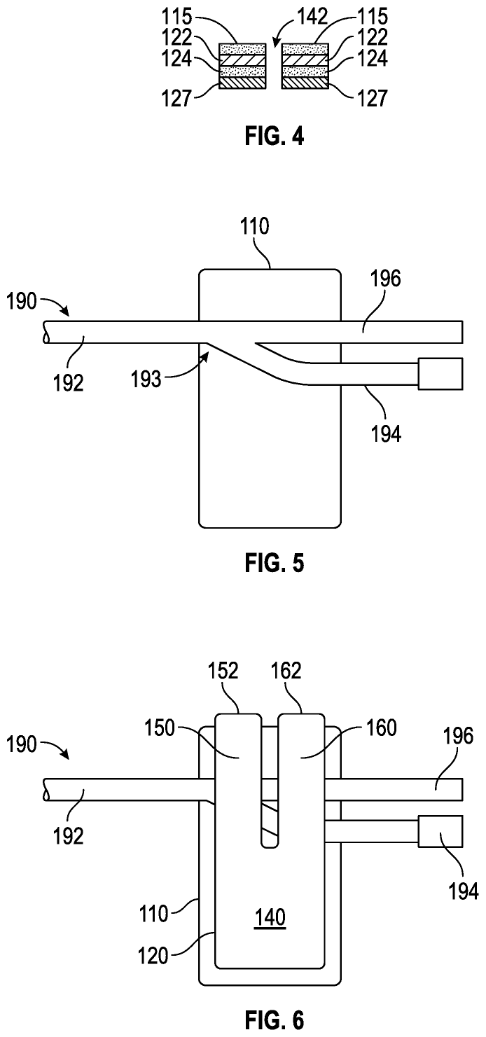

The depicted illustrative embodiment of coupling article 120 as depicted in FIG. 1 is also depicted in an exploded cross-sectional view in FIG. 3 (where the cross-section is taken along line 3-3 in FIG. 1), as well as in a cross-sectional view in FIG. 4 (where the cross-section is taken along line 4-4 in FIG. 1). The depicted embodiment of coupling article 120 as depicted in the exploded cross-sectional view of FIG. 3, can be configured (e.g., dimensioned, shaped, formed of appropriate materials, etc.) to be adhered to the base articles 110 after the base articles 110 have been attached to the skin of a patient at a selected location, e.g., on a leg, arm, torso, etc.

The depicted illustrative embodiment of coupling article 120 includes an anchor section 140 along with first and second legs 150 and 160 extending away from the anchor section 140. In one or more embodiments, the first and second legs 150 and 160 may be collectively referred to as a capture section 180 which is attached to the anchor section 140 in the depicted illustrative embodiment of coupling article 120. The first leg 150 includes a first distal end 152, with the first leg 150 extending away from the anchor section 140 to the first distal end 152 of the first leg 150 along a first direction as defined by axis 151 in FIG. 1. The second leg 160 includes a second distal end 162, with the second leg 160 extending away from the anchor section 142 the second distal end 162 of the second leg 150 along a second direction as defined by axis 161 in FIG. 1.

In one or more embodiments, the axes 151 and 161 that define the directions along which the first and second legs 150 and 160 extend away from the anchor section 140 may be aligned with each other which, in one or more embodiments, may mean that the axes 151 and 161 are parallel with each other although the axes need not be perfectly parallel, i.e., they may, in one or more embodiments, diverge or converge. Further, because the first and second legs 150 and 160 have a width measured generally transverse to their respective axes, the axes along which each of the first and second legs 150 and 160 extends may be parallel with each other, while the legs 150 and 160 may appear to diverge or converge when moving away from the anchor section 140.

Another optional feature depicted in connection with coupling article 120 as seen in FIG. 1 is the gap 142 located between first leg 150 and second leg 160. In one or more embodiments, a gap 142 may be useful to assist in separate manipulation of the legs 150 and 160 during attachment of a catheter using the catheter securement systems described herein.

In the depicted illustrative embodiment as seen in FIG. 3, the coupling article 120 includes a backing layer 122 on which securing adhesive 124 is located. In particular, the backing layer 122 provides a first (e.g., bottom) major surface 121 on which securing adhesive 124 is located, along with a second (e.g., top) major surface 123 opposite the first major surface 121. Although the backing layer 122 of coupling article 120 is depicted as being in the form of a single unitary layer, the backing layer 122 may, in one or more embodiments, include multiple layers and/or components as desired.

While only one securing adhesive 124 is shown as being present on the first major surface of the coupling article 120, in one or more embodiments of coupling articles of catheter securement systems as described herein, different portions of the coupling article 120 may include different securing adhesives located thereon. For example, in one or more embodiments, the anchor section 140 may have a first securing adhesive while one or both of the legs 150 and 160 may have a different securing adhesive. The securing adhesive in the anchor section 140 of the coupling article 120 may be less aggressive with a lower peel force on the base article 110 than the securing adhesive used on the legs 150 and 160, which may be more aggressive to provide a higher peel force on the outer surface of the catheter tubes to be attached to the base articles 110. In still other alternative embodiments, the aggressiveness of the adhesives on the anchor sections 140 and legs 150 or 160 of coupling articles 120 may be reversed, i.e., the securing adhesive on the anchor section 140 may be more aggressive while the securing adhesive on the legs 150 and 160 may be less aggressive. In still other embodiments, portions of the coupling article 120 may be free of adhesive such that the coupling article 120 does not adhesively attached to the catheter tubes.

The depicted embodiment of coupling article 120 also includes a release liner 126 covering securing adhesive 124 until application of the coupling article 120 on a base article 110 as described herein is desired. In particular, the depicted embodiment of release liner 126 is provided in multiple parts to allow for selective exposure of securing adhesive 124 on various portions of the coupling article 120, to in one or more embodiments, assist in proper application of the coupling article over a catheter on a backing article 110 as described herein. Alternatively, however, a release liner provided on a coupling article 120 may be in the form of a single unitary article removed to expose all of the securing adhesive 124 on a coupling article 120 at the same time.

In those embodiments in which the coupling article 120 includes a release liner 126 provided in multiple parts, the securing adhesive 124 on different portions of the coupling article 120 may be selectively exposed where the different parts of the release liner 126 are independently removable. For example, in one or more embodiments in which the anchor section 140 includes a release liner 125 separate from the release liners 127 on the first and second legs 150 and 160, the anchor section release liner 125 may be configured for removal from the anchor section 140 independently of removal of the first leg release liner 127 from the first leg 150 as well as being configured for removal independently of the second leg release liner 127 from the second leg 160. Further, where separate release liners 127 are provided on the first and second legs 150 and 160, the release liners 127 on the legs 150 and 160 may be removed from their respective leg independently of removal of the release liner on the other leg.

Although the release liner 126 is not depicted on the kit sheet 102 in FIG. 3, all or a portion of the release liner 126 may, in one or more embodiments, be provided as a portion of the kit sheet 102 as depicted in FIG. 1. For example, the anchor section portion 125 of release liner 126 may be provided on kit sheet 102, while the leg portions 127 of release liner 126 may be provided as separate articles that are not attached to the kit sheet 102. In such embodiments, the coupling article 120 can be removed from the kit sheet 102 to expose the securing adhesive 124 on the anchor section 140 while the securing adhesive 124 on each of the first and second legs 150 and 160 remains covered by leg portions 127 of release liner 126.

Another optional feature of release liners that may be used on coupling articles of catheter securement systems as described herein are tabs 128 located at the junctions between the anchor section portion 125 of release liner 126 and the leg portions 127 of release liner 126 as seen in, e.g., FIG. 3. Tabs 128 may be, in one or more embodiments, be formed by overlapping release liner sections and/or folding one or more of the release liner sections. In particular, leg portions 127 of release liner 126 are depicted as having a fold which forms tab 128 of leg section 127 of release liner 126.

One illustrative embodiment of a method of using coupling article 120 along with base articles 110 to secure a catheter on a selected portion of the skin of a patient is depicted in FIGS. 5-8. In particular, FIG. 5 depicts a base article in the form of backing layer 112 after attachment to the skin of a patient and after removal of the tabs 118 from the backing layer 112.

The depicted illustrative embodiment of catheter 190 to be secured using one illustrative embodiment of a catheter securement system as described herein is in the form of a bifurcated catheter 190 having a Y junction 193 at which a supply tube 192 bifurcated into a first branch tube 194 and a second branch tube 196.

With the catheter 190 in position such that the Y junction 193 is located over the base article 110, the anchor section 140 of a coupling article 120 is attached to the base article 110 adjacent the Y junction 193 as depicted in, e.g., FIG. 6. In one or more embodiments, the legs 150 and 160 of the coupling article 120 may be positioned over the catheter 190, such that the tubes of the catheter 190 can be described as being located between the legs 150 and 160 of the coupling article 120 and the base article 110 attached to the skin of a patient.

Although the coupling article 120 is described as being attached to the base article 110 after positioning the catheter 190 on the base article 110, one or more alternative embodiments of methods as described herein may include attaching the anchor section 140 of a coupling article 120 to the base article 110 before positioning the catheter 190 on the base article 110.

Regardless of the order in which the anchor section 140 of coupling article 120 is attached to base article 110 and the catheter 190 is positioned on the base article 110, placement of the coupling article 120 and positioning of the catheter 190 preferably results in the arrangement as depicted in FIG. 6 in which the legs 150 and 160 of the coupling article 120 are positioned over the catheter 190, such that the tubes of the catheter 190 can be described as being located between the legs 150 and 160 of the coupling article 120 and the base article 110.

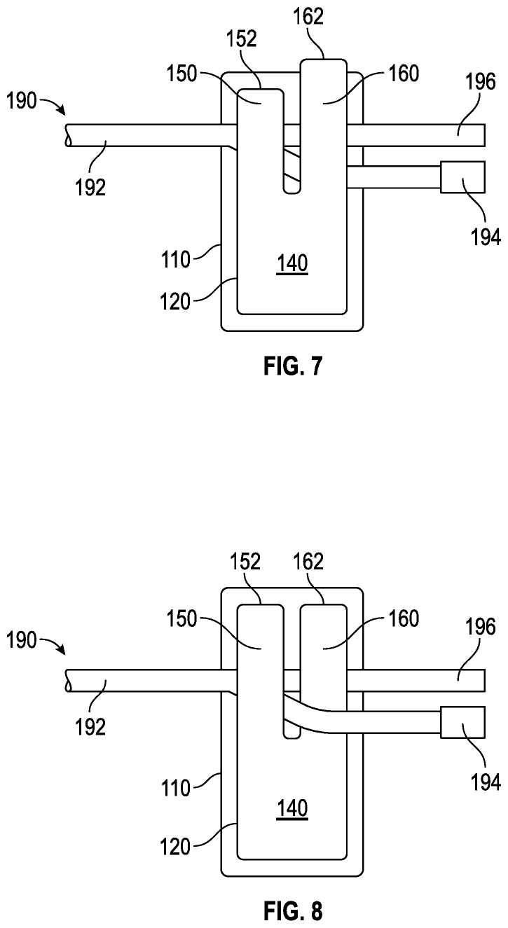

With the components located as depicted in FIG. 6, the distal end 152 of leg 150 of coupling article 120 is attached to base article 110 such that the supply tube 192 is located between the anchor section 140 and the distal end 152 of the leg 150, with the intermediate portion of leg 150 wrapping over the supply tube 192 as depicted in FIG. 7. The leg 150 as depicted in FIG. 7 appears shorter than leg 150 as depicted in FIG. 6 because the leg 150 is wrapped over the supply tube 192. Attachment of the distal end 152 of the leg 150 to the base article 110 may, in one or more embodiments, involve removal of a leg section 127 of release liner 126 (see, e.g., FIG. 3) from the leg 150.

After attachment of the distal end 152 of leg 150 to the base article 110 such that leg 150 is wrapped over the supply tube 192, the distal end 162 of leg 160 of coupling article 120 may also be attached to the base article 110 as depicted in FIG. 8. Attachment of the distal end 162 of leg 160 to the base article 110 as depicted in FIG. 8 involves locating the first branch tube 194 of the catheter 190 over a portion of the second leg 160 while locating the second branch tube 196 of the catheter 190 between the second leg 160 and the base article 110. As a result, the portion of the second leg 160 located closer to the anchor section 140 than the distal end 162 is located between the first branch tube 194 of the catheter 190 and the base article 110. Further, the portion of the second leg 160 located closer to the distal end 162 than the anchor section 140 is located over the second branch tube 196 of the catheter 190 and may be described as being wrapped over the second branch tube 196 of the catheter 190 such that the second branch to 196 is located between the second leg 160 and the base article 110. Attachment of the distal end 162 of the leg 160 to the base article 110 may, in one or more embodiments, involve removal of a leg section 127 of release liner 126 (see, e.g., FIG. 3) from the leg 160.

In one or more embodiments, the Y junction 193 of the catheter 190 may be positioned as such that the point at which the first and second branch tubes 194 and 196 diverge is located between the legs 150 and 160 of the coupling article 120. Such an arrangement may facilitate manipulation of the second leg 160 relative to the first and second branch tubes to achieve the woven or interleaved relationship as depicted in FIG. 8. Further, positioning one leg 150 of the coupling article 120 on one side of the Y junction 193 while positioning the second leg 160 in a woven or interleaved relationship with the branch tubes 194 and 196 on the opposite side of the Y junction may assist in securing the catheter 190 in position on the base article 110 due to mechanical interference between the legs 150 and 160 and the catheter 190.

Although the method as described above in connection with FIGS. 5-8 involves placement of the leg 150 before placement of the leg 160, it should be understood that in one or more alternative embodiments of methods as described herein the legs 150 and 160 may be attached to the base article in any order to secure a catheter 190 on the base article 110.

As discussed herein, the catheter securement systems may advantageously provide for repositioning of the catheter 190 from one base article 110 two another base article 110 positioned at a different location on a patient. In one or more embodiments of the methods as described herein, the same coupling article 120 may be used to attach the catheter 190 to a second base article 110. Repositioning of catheter 190 in a method that involves reusing coupling article 120 involves detaching the legs 150 and 160 and the anchor section 140 from a first base article 110 and moving the catheter 190 along with coupling article 120 into a position above a second base article 110, followed by reattachment of the distal ends 152 and 162 of the legs of coupling article 120 along with the anchor section 140 of the coupling article 120 to the second base article 110.

Moving a coupling article 120 of a catheter securement system as described herein from a first base article 110 to a second base article 110 may be facilitated, in one or more embodiments, where the base articles 110 include a surface that allows for removal of the securing adhesive. In one or more embodiments, the base articles 110 of one or more embodiments of catheter securement systems as described herein may include a release agent on their second major surfaces 113. With reference to the illustrative embodiment of base article 110 as depicted in FIG. 2, the second major surface 113 of the backing layer 112 may include a low adhesion (low adhesion backsize, or LAB) coating 115 at least in those regions positioned to come into contact with the securing adhesive 124 of coupling article 120. Such a coating 115 can allow the coupling article 120 to be removed from and/or reattached to base articles 110 at different locations on a patient as necessary. A description of a low adhesion backing material that may be suitable for use with the base articles 110 and coupling articles 120 as described herein can be found in U.S. Pat. Nos. 5,531,855 and 6,264,976.

Although the coupling article 120 described in connection with FIGS. 1-8 is repositionably attached to the base articles 110 using a securing adhesive, one or more alternative embodiments of coupling articles and base articles of catheter securement systems as described herein may use other types of repositionable connections between the coupling article and the base articles.

One alternative embodiment of a base article and associated coupling article that may be used in one or more embodiments of catheter securement systems as described herein may be in the form of non-tacky/cohesive polymeric materials that allow for repositioning of articles after an initial attachment.

For example, a layer of non-tacky/cohesive polymeric material may be provided on the coupling articles 120 in place of a pressure sensitive adhesive 124 as described herein. Backing layer 112 of the base article 110 may, in one or more embodiments, present a surface to which the non-tacky/cohesive polymeric material on the coupling article 120 attaches without the addition of a selected attachment layer or other treatment. In one or more alternative embodiments, the backing layer 112 of the base article 110 may include target materials (e.g., a layer, film, etc.) to which the non-tacky/cohesive polymeric material on the coupling article 120 attaches. In still one or more other alternative embodiments, a layer of non-tacky/cohesive polymeric material may be provided on the second major surface 113 of the backing layer 112 of the base article 110 (in place of or in addition to, e.g., release coating 115) while the coupling article 120 includes materials to which the non-tacky/cohesive polymeric material on the base article 110 attaches.

In all instances, the non-tacky/cohesive polymeric materials used on one or both of the coupling articles and the base articles of catheter securement systems as described herein provide for repositionable attachment of the coupling articles 120 on base articles 110 as described herein. Examples of some potentially suitable non-tacky/cohesive polymeric materials or attachment systems that may provide for repositionable attachment of the coupling articles 120 on the base articles as described herein may be described in, e.g., EP 0443263 and/or U.S. Pat. Nos. 5,888,335; 5,908,695; 6,004,670; and 7,135,213.

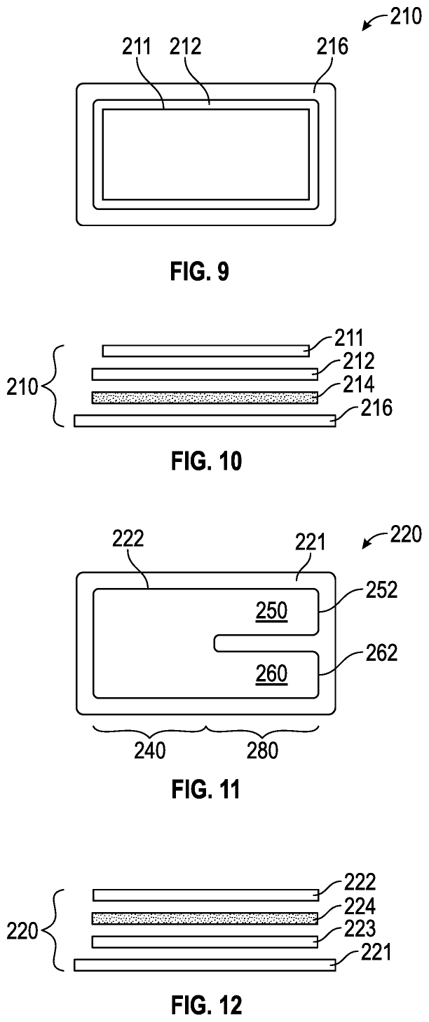

In still other alternative embodiments of catheter securement systems as described herein, the coupling articles and base articles may be repositionably attached to each other using mechanical fasteners. One illustrative embodiment of a base article 210 and a complementary coupling article 220 using mechanical fasteners for repositionable attachment are depicted in FIGS. 9-12.

In particular, FIG. 9 is a plan view of one alternative embodiment of a base article 210, while FIG. 10 is an exploded edge view of the base article 210 depicting various components found in the depicted illustrative embodiment of base article 210. The base article 210 includes, in the depicted illustrative embodiment, skin-contact adhesive 214 on a backing layer 212. A release liner 216 may be provided to protect the skin-contact adhesive 214 on backing layer 212 as discussed in connection with other alternative embodiments of base articles as described herein.

In the depicted illustrative embodiment, the base article 210 also includes a mechanical fastener component 211 attached to backing layer 212. In one or more embodiments, the mechanical fastener component 211 may be attached to backing layer 212 by any suitable technique or combination of techniques. For example, in one or more embodiments, the mechanical fastener component 211 may be attached to backing layer 212 using one or more adhesives, thermal welding, ultrasonic welding, chemical welding, sewing, etc.

Although the backing layer 212 and mechanical fastener component 211 are depicted as separate articles, the backing layer 212 may function as a mechanical fastener component 211 in addition to providing a backing layer in one or more alternative embodiments of base articles 210 as described herein. In many instances, however, the backing layer 212 may present a softer, more flexible and conformable surface which may be useful in attaching base article 210 to the skin of a patient. Further, the backing layer 212 may extend beyond the edges of the perimeter of mechanical fastener component 211 to protect the skin of a patient where, for example, the mechanical fastener component 211 may present a stiffer or potentially irritating edge from which protection is desired.

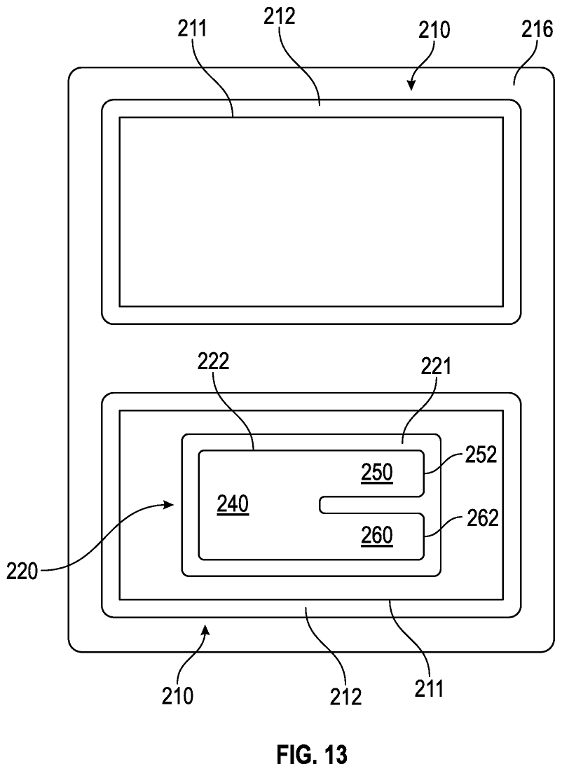

Referring to FIGS. 11-12, one illustrative embodiment of a coupling article 220 that may be repositionably attached to the mechanical fastener component 211 of the base article 210 is depicted. Coupling article 220 may include a backing layer 222 having a securing adhesive 224 located on a first major surface of the backing layer 222. The backing layer 222 of coupling article 220 is attached to a carrier 223 using securing adhesive 224. The depicted illustrative embodiment of coupling article 220 further includes a mechanical fastener component 221 attached to the opposite side of carrier 223 and is configured to attached to the mechanical fastener component 211 of base article 210.

Catheter securement systems having coupling articles that include a carrier such as the illustrative embodiment of coupling article 220 with carrier 223 (and its associated mechanical fastener component 221) may offer advantages during use. For example, a catheter may remain captured by the coupling article 220 with only the carrier 223 being detached from the base article 210 when moving from one base article to a different base article as described herein.

The backing layer 222 and its securing adhesive 224 may, in one or more embodiments, be attached directly to the mechanical fastener component 221. In the depicted illustrative embodiment, however, the backing layer 222 and securing adhesive 224 attached to a carrier 223 that is, itself, attached to the mechanical fastener component 221. The carrier 223 may provide a surface that provides more secure attachment of securing adhesive 224 than the mechanical fastener component 221 and, as discussed herein, may allow for a catheter captured by the coupling article 220 remain captured by the coupling article 220 during transfers of the catheter and coupling article between different base articles as described herein.

In the depicted illustrative embodiment, securing adhesive 224 is located on both the anchor section 240, as well as the legs 250 and 260 of the backing layer 222 of the coupling article 220. In one or more embodiments, the legs 250 and 260 may be collectively referred to as a capture section 280 which is attached to the anchor section 240 in the depicted illustrative embodiment of coupling article 220.

In one or more alternative embodiments, however, the anchor section 240 of the coupling article 220 may be directly attached to the carrier 223 or to the mechanical fastener component 221, while the legs 250 and 260 are used to secure a catheter to the base article 210 using adhesive attachment.

FIG. 13 depicts one illustrative embodiment of a catheter securement system including a pair of base articles 210 as described in connection with FIGS. 9 and 10 along with a coupling article 220 as described in connection with FIGS. 11 and 12. The base articles 210 as seen in FIG. 13 are attached to a release liner 216 which functions as a kit sheet as discussed above in connection with, e.g., FIG. 1. In one or more embodiments, the release liner/kit sheet 216 may include a release surface configured to allow for removal of the base articles 210 from the release liner/kit sheet 216 as described herein.

Another feature which may be provided in one or more embodiments of catheter securement systems as described herein, a coupling article 220 is depicted as being attached to one of the base articles 210. In other words, the coupling article 220 can be described as being pre-assembled with one of the base articles 210 in contrast to the catheter securement system as depicted in, e.g., FIG. 1, where the coupling article 120 is separately attached to a kit sheet 102. A coupling article with a base article in one or more embodiments of catheter securement systems as described herein may reduce the number of steps required for a user to employ the catheter securement systems to secure a catheter on a patient.

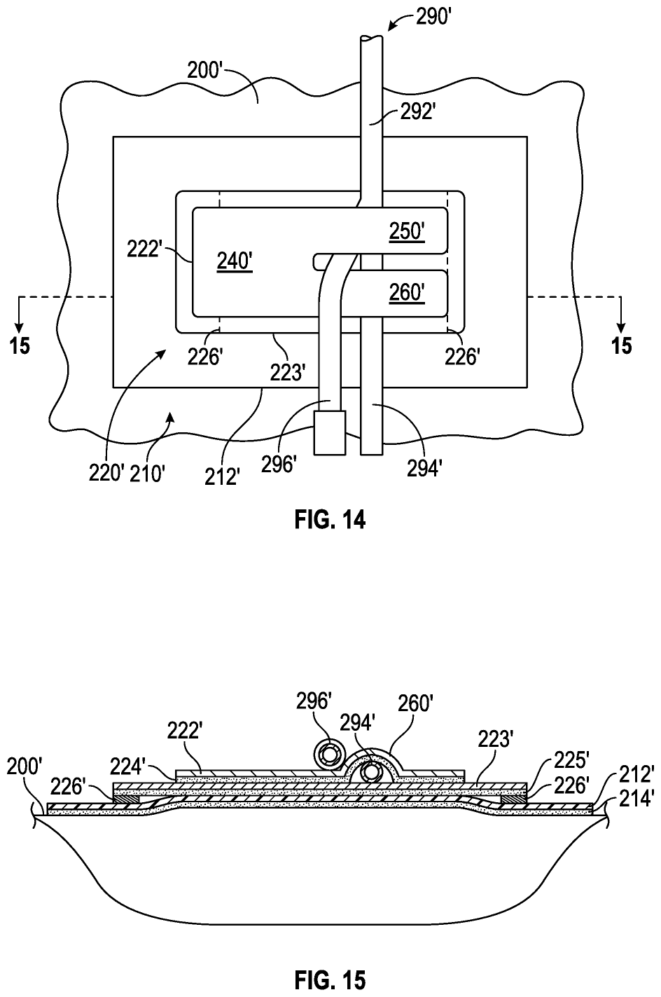

Although the illustrative embodiment of the catheter securement system depicted in connection with FIGS. 9-13 use mechanical fasteners to attach the carrier 223 of a coupling article 220 to one or more base articles 210, other alternative embodiments of catheter securement systems as described herein may use pressure sensitive adhesives or non-tacky/cohesive polymeric materials to attach a carrier of a coupling article to a base article. One illustrative embodiment of a catheter securement system using a pressure sensitive adhesive or non-tacky/cohesive polymeric material to attach a carrier of a coupling article to a base article is depicted in connection with FIGS. 14 and 15.

FIG. 15 is a cross-sectional view of the catheter securement system as seen in the plan view of FIG. 14 with the cross-sectional view taken along line 15-15 in FIG. 14. The catheter securement system includes a base article 210′ and a coupling article 220′ attached to the base article 210′. The base article 210′ is, in the depicted illustrative embodiment, attached to the skin 200′ of a patient.

The catheter securement system of FIGS. 14 and 15 is depicted as having secured a catheter 290′ to the skin 200′ of the patient using the base article 210′ and the coupling article 220′ attached to the base article 210′. The catheter 290′ includes a supply tube 292′ and branch tubes 294′ and 296′ that connect to the supply tube 292′ at a Y junction in a manner similar to other catheters described herein. In particular, the coupling article 220′ includes a backing layer 222′ having an anchor section 240′ along with legs 250′ and 260′. The anchor section 240′ and at least the ends of the legs 250′ and 260′ are attached to the carrier 223′.

In the depicted illustrative embodiment, the carrier 223′ is attached to the backing layer 212′ of the base article 210′ using carrier adhesive 225′. Transfers of the catheter 290′ along with the coupling article 220′ from one base article to another base article as described herein, may, in one or more embodiments, be facilitated by tabs 226′ provided on the carrier 223′ such that the carrier adhesive 225′ does not adhere the carrier 223′ completely to the backing layer 212′ of the base article 210′. In particular, the tabs 226′ in the depicted embodiment of coupling article 220′ are in the form of bond block layers adhered to the carrier adhesive 225′ in the areas of tabs 226′. Many other alternative constructions for tabs and other features that enhance removal of adhesive layers from surfaces may be used in place of the bond block features 226′ used to form tabs 226′.

Although the illustrative embodiment of carrier 223′ of coupling article 220′ is described above as being attached to the backing layer 212′ of base article 210′ using adhesive, one or more layers of non-tacky/cohesive polymeric materials may be used in place of adhesives as described herein to attach the carrier 223′ to the backing layer 212′ of the base article 210′.

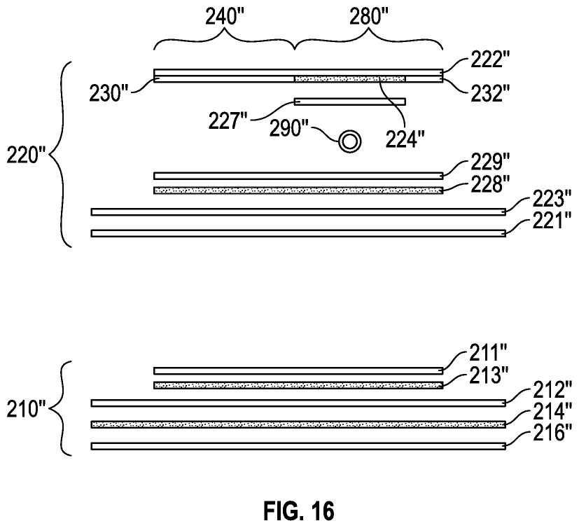

Another illustrative embodiment of a catheter securement system including a base article 210″ and a coupling article 220″ is depicted in an exploded edge view in FIG. 16. In the illustrative embodiment of coupling article 220″, a combination of mechanical fasteners and adhesives and/or non-tacky/cohesive polymeric materials may be used to secure a catheter tube 290″.

The base article 210″ includes a backing layer 212″ and a skin-contact adhesive 214″ used to secure the base article 210″ to the skin of a patient as described herein. In one or more embodiments, the skin-contact adhesive 214″ may be protected by a liner 216″ which can be removed to attach the base article 210″ to the skin of a patient. In those embodiments in which mechanical fasteners are used to attach a coupling article such as, e.g., coupling article 220″, to the backing article 210″, the base article 210″ may also include a mechanical fastener component 211″. The mechanical fastener component 211″ may, in one or more embodiments, be attached to the backing layer 212″ using adhesive 213″, although other techniques for attaching mechanical fastener components to backing layers may be used as described herein.

The depicted illustrative embodiment of coupling article 220″ includes a carrier 223″ and a mechanical fastener component 221″ as separate components, although it should be understood that in one or more embodiments of coupling articles as described herein, the carrier and a mechanical fastener component may be provided as a unitary article where the mechanical fastener component provides a suitable surface for attachment of other components of coupling articles as described herein. In the depicted illustrative embodiment, mechanical fastener component 221″ is complementary to the mechanical fastener component 211″ on the base article such that the coupling article 220″ can be repositionably attached to the base article 210″ as described herein.

In one or more alternative embodiments where the base article does not include a mechanical fastener component 211″, the mechanical fastener component 221″ of illustrative embodiment of coupling article 220″ may be replaced by, e.g., pressure sensitive adhesive and/or non-tacky/cohesive polymeric materials as described herein.

The depicted illustrative embodiment of coupling article 220″ includes a combination of materials used to secure a catheter in and/or on the coupling article 220″. The coupling article 220″ depicted in FIG. 16 includes a backing layer 222″ having an anchor section 240″ along with a capture section 280″. The backing layer 222″ includes, in the depicted embodiment, securing adhesive 224″ as well as mechanical fastener components 230″ and 232″.

The securing adhesive 224″ may be used to adhere to a catheter tube 290″ captured within the coupling article 220″. In the depicted illustrative embodiment, the coupling article 220″ includes a release liner 227″ which can be removed before securing a catheter tube 290″ in the coupling article 220″.

Unlike the embodiment of coupling article 220′ as depicted in FIGS. 14 and 15, the remainder of the backing layer 222″ is attached to the remainder of the coupling article 220″ using mechanical fastener components 230″ and 232″. Coupling article 220″ also includes a complementary mechanical fastener component 229″ positioned beneath the backing layer 222″ such that the mechanical fastener components 230″ and 232″ on the backing layer 222″ can be repositionably attached to the carrier 223″.

In the depicted illustrative embodiment of coupling article 220″, the mechanical fastener component 229″ located between the carrier 223″ and the backing layer 222″ may be attached to the carrier using a securing adhesive 228″, although other techniques of attaching mechanical fastener components to other components within coupling articles as described herein may be used. Furthermore, in one or more alternative embodiments, the carrier 223″ may include a surface that is, itself, a mechanical fastener component. In such embodiments, a separate mechanical fastener component 229″ and any related adhesive 228″ may not be required.

Another characteristic feature of one or more embodiments of catheter securement systems as described herein may be the relative sizes of the coupling articles and the base articles. The base articles of one or more embodiments of catheter securement systems as described herein can be described as having a base article footprint area, which is measured when the base article is in a flat configuration as shown in, e.g., the illustrative embodiment of base articles 210 in FIG. 13. This base article area is the portion that each of the base articles 210 occupies on the release liner/kit sheet 216 and generally corresponds to the area occupied by one of the base articles 210 on the skin of a patient.

The coupling articles of one or more embodiments of catheter securement systems as described herein can be described as having a coupling article footprint area when measured with the coupling article in a flat configuration as also shown in, e.g., the illustrative embodiment of coupling article 220 as seen in FIG. 13.

In one or more embodiments of the catheter securement systems described herein, the coupling articles may have a coupling article footprint area that is less than the base article footprint area used with the coupling articles. Such an arrangement may facilitate easier placement of the coupling articles on the base articles in catheter securement systems as described herein. In one or more embodiments, the coupling article footprint area occupied by a coupling article on a base article may be 90% or less; 80% or less, 70% or less, or 60% or less of the base article footprint area. At the lower end of the range, it may be preferred that the coupling article footprint area, i.e., the area of a base article that is occupied by a coupling article located thereon may be 10% or more, 20% or more or 30% or more of the base article footprint area.

Regardless of the relative sizes of the base article footprint area and the coupling article footprint area of catheter securement systems as described herein, the size of the coupling article footprint areas of coupling articles described herein are preferably large enough to resist unwanted detachment of the coupling articles from base articles to which they are attached to secure a catheter at a selected location on a patient.

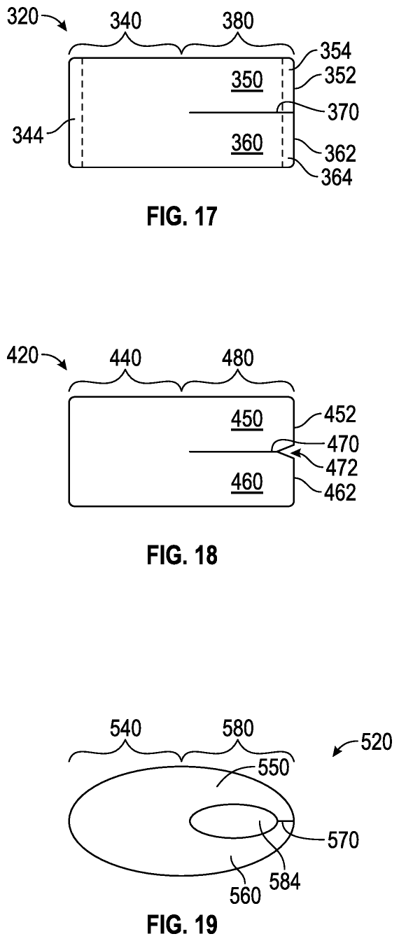

FIGS. 17-19 depict three alternative illustrative embodiments of coupling articles that may be used in one or more embodiments of catheter securement systems as described herein. As discussed above in connection with coupling article 120, the legs 150 and 160 in the capture section 180 of coupling article 120 are separated from each other by a gap 142. In the alternative illustrative embodiment of coupling article 320 as depicted in FIG. 17, the first leg 350 and second leg 360 of the capture section 380 attached to anchor section 340 may be constructed from a backing layer that includes a line of separation 370 formed in the backing layer that defines the first leg 350 and the second leg 360 of the coupling article 320. In the depicted embodiment, the line of separation 370 extends from the anchor section 344 the first and second distal ends 352 and 362 of the first and second leg 350 and 360. The line of separation 370 may take a variety of different forms (e.g., perforations, a line of weakness, etc.) that allow the backing layer to preferentially separate along the line of separation 370 when subjected to tension forces across the line of separation 370.

Other optional features depicted in connection with the illustrative embodiment of coupling article 320 are tabs on various portions of the coupling article 320. In particular, the anchor section 340 may include one or more tabs such as, e.g., tab 344, while each of the leg 350 and 360 may include tabs 354 and 364, respectively. The tabs on the various portions of the coupling article 320 may improve the ability of a user to manipulate the various portions of the coupling article 320 during initial placement on a base article and/or during repositioning of the coupling article 320 from one base article to another base article.

In one or more embodiments, the tabs 344, 354, and/or 364 may be formed by areas in which securing adhesive is not provided, by covering or otherwise interfering with the ability of the securing adhesive located on the coupling article 322 attached to a base article, etc. As a result, the portions of any backing layer located within the tabs will not typically be adhered to the base article which may enhance the ability of a user to grasp the tabs.

Another alternative illustrative embodiment of a coupling article 420 is depicted in FIG. 18, where coupling article 420 includes an anchor section 440 to which capture section 480 is attached. Capture section 480 includes 450 and 460 extending away from the anchor section 440. The legs 450 and 460 may be separated from each other along a line of separation 470 as discussed above in connection with coupling article 320. The coupling article 420 also includes a notch 472 formed in the backing layer proximate the distal ends 452 and 462 of the legs 450 and 460. The notch 472 may facilitate separation of a backing layer along line of separation 470 by a user.

Yet another alternative illustrative embodiment of a coupling article 520 is depicted in FIG. 19, where coupling article 520 includes an anchor section 540 to which capture section 580 is attached. Capture section 580 includes legs 550 and 560 that extend away from the anchor section 540. In this illustrative embodiment, the legs 550 and 560 are separated from each other along a majority of their length by an aperture 584 formed between the legs 550 and 560. The depicted illustrative embodiment of coupling article 520, however, may include a line of separation 570 across which the legs 550 and 560 may be attached to each other proximate the ends of the legs 550 and 560.

Retaining a connection between legs 550 and 560 proximate the distal ends of the legs may facilitate handling of the coupling article 520 during initial placement of the coupling article 520 on a base article. More specifically, the anchor section 540 may be more easily attached to a base article when the legs 550 and 560 are connected to each other at their distal ends. Following attachment of the anchor section 542 a base article, the legs 550 and 560 may be separated from each other along line of separation 570 to allow for securement of a catheter as described herein in connection with, e.g., FIGS. 5-8.

Another illustrative embodiment of a coupling article for use in one or more embodiments of a catheter securement system as described herein depicted in FIG. 20A. The depicted embodiment of coupling article 620 includes a carrier 623 similar to other coupling articles with carriers as described herein. As discussed herein, carriers on coupling articles in one or more embodiments of catheter securement systems may be repositionably attached to base articles of the catheter securement systems using one or more of pressure sensitive adhesives, non-tacky/cohesive polymeric materials, and mechanical fastener components. In addition, carrier 623 may, in one or more embodiments, include one or more tabs such as, e.g., tab 626 depicted in FIG. 20A to facilitate placement and/or removal of the carrier from a base article in a catheter securement system as described herein.

The depicted embodiment of coupling article 620 further includes two coupling article components 670a and 670b, each of which includes its own anchor section 640a or 640b. Coupling article component 670a includes an anchor section 640a and a leg 650a extending away from the anchor section 640a. Leg 650a may include an end located distal from the anchor section 640a and, in one or more embodiments, the end of leg 650a may include a tab 654a which may be used to facilitate manipulation of the leg 650a during capture of a catheter on the coupling article 620.

Coupling article component 670b includes an anchor section 640b and a leg 650b extending away from the anchor section 640b. Leg 650b may also include an end located distal from the anchor section 640b and, in one or more embodiments, the end of the leg 650b may include a tab 654b which may be used to facilitate manipulation of the leg 650b during capture of a catheter on the coupling article 620.

In one or more embodiments of coupling article 620 including two or more coupling article components such as those depicted in FIG. 20A, the anchor sections 640a and 640b of two or more of the coupling article components may be located adjacent each other as depicted in connection with coupling article 620 of FIG. 20A. Such arrangements may, however, not be required in other alternative embodiments of coupling articles including carriers as described herein.

Although one or more embodiments of coupling articles which may be used in one or more embodiments of catheter securement systems described herein may include separate and distinct coupling article components such as those depicted in FIG. 20A, the use of a common anchor section such as the common anchor sections from which multiple legs extend in other alternative embodiments of coupling articles as described herein may provide advantages in improving retention forces for catheters captured by a coupling articles including common anchor sections having two or more legs extending therefrom.

Still another illustrative embodiment of a coupling article that may be used in one or more embodiments of catheter securement systems as described herein is depicted in FIG. 20B. The depicted embodiment of coupling article 720 includes a carrier 723 similar to other coupling articles with carriers as described herein. As discussed herein, carriers on coupling articles in one or more embodiments of catheter securement systems may be repositionably attached to base articles of the catheter securement systems using one or more of pressure sensitive adhesives, non-tacky/cohesive polymeric materials, and mechanical fastener components. In addition, carrier 723 may, in one or more embodiments, include one or more tabs such as, e.g., tab 726 depicted in FIG. 20B to facilitate placement and/or removal of the carrier from a base article in a catheter securement system as described herein.

The depicted embodiment of coupling article 720 further includes two coupling article components 770a and 770b, each of which includes its own anchor section 740a or 740b. Coupling article component 770a includes an anchor section 740a and a leg 750a extending away from the anchor section 740a. Leg 750a may include an end located distal from the anchor section 740a and, in one or more embodiments, the end of leg 750a may include a tab 754a which may be used to facilitate manipulation of the leg 750a during capture of a catheter on the coupling article 720.

Coupling article component 770b includes an anchor section 740b and a leg 750b extending away from the anchor section 740b. Leg 750b may also include an end located distal from the anchor section 740b and, in one or more embodiments, the end of the leg 750b may include a tab 754b which may be used to facilitate manipulation of the leg 750b during capture of a catheter on the coupling article 720.

In one or more embodiments of coupling article 720 including two or more coupling article components such as those depicted in FIG. 20B, the anchor section of one of the coupling article components may be located proximate the end of the leg of another coupling article component. In the depicted illustrative embodiment of coupling article 720, the anchor section 740a of coupling article component 770a is located proximate the end of the leg 750b of coupling article component 770b. In addition, the depicted illustrative embodiment of coupling article 720 depicts an arrangement in which the anchor section 740b of coupling article component 770b is located proximate the end of the leg 750a of coupling article component 770a.

Another optional feature of one or more embodiments of coupling articles that may be used in one or more embodiments of catheter securement systems as described herein that is depicted in connection with coupling article 720 of FIG. 20B are the enlarged anchor sections 740a and 740b of the different coupling article components 770a and 770b.

Further, although the depicted illustrative embodiments of coupling articles depicted in FIGS. 20A-20B include two coupling article components mounted on the same carrier, it should be understood that one or more alternative embodiments of coupling articles as described herein may include only one coupling article component on a carrier, i.e., an anchor section having only one leg extending therefrom. Furthermore in still other alternative embodiments, coupling articles used in one or more embodiments of catheter securement systems as described herein may include three or more coupling article components on a carrier as needed or desired to secure a catheter on a patient as described herein.

In yet other alternative embodiments, coupling articles used in one or more embodiments of catheter securement systems as described herein may include one or more coupling article components that include a common anchor section having two or more legs extending therefrom (as described herein in connection with other embodiments of coupling articles) and one or more additional coupling article components having a single anchor section with a single leg extending therefrom (as depicted in connection with, e.g., FIGS. 20A-20B), with all of the coupling article components mounted on the same carrier.

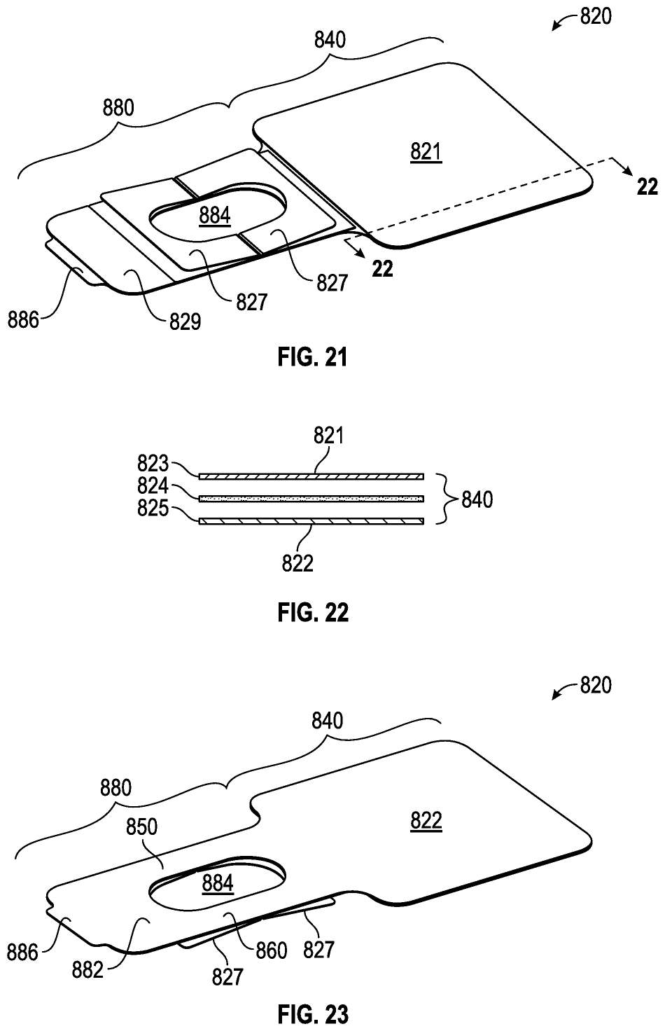

Yet another illustrative embodiment of a catheter securement system as described herein is depicted in connection with FIGS. 21-25. The catheter securement system depicted in those figures includes a coupling article 820 along with one or more base articles 810 to which the coupling articles may be repositionably attached as described herein.

Referring to FIGS. 21-23, the coupling article 820 includes an anchor section 840 and a capture section 880 attached to the anchor section 840. The anchor section 840 includes a first major surface 821 and a second major surface 822 on an opposite side of the anchor section 840 from the first major surface 821.

In one or more embodiments, the anchor section 840 may include multiple components with one illustrative embodiment of a set of components found in the depicted illustrative embodiment of anchor section 840 being depicted in the cross-sectional view of FIG. 22 which is taken along line 22-22 as seen in FIG. 21. The components found in the illustrative embodiment of anchor section 840 may include a carrier 823 forming the first major surface 821 with a backing layer 825 forming the opposite second major surface 822. The carrier 823 may be attached to the backing 825 by, in the depicted illustrative embodiment, a layer of adhesive 824.

In one or more embodiments, the carrier 823 may provide a first major surface 821 that includes a securing adhesive or a non-tacky/cohesive polymeric material used to attach the anchor section to a base article 810 (see, e.g., FIG. 22) in a manner similar to the coupling article 220′ depicted in FIGS. 14-15.

In one or more alternative embodiments, the carrier 823 may provide a first major surface 821 that includes a mechanical fastener component that attaches to a complementary mechanical fastener component provided on the base article 810 in a manner similar to coupling article 220 depicted in FIGS. 9-13.

The depicted illustrative embodiment of coupling article 820 also includes a capture section 880 including legs 850 and 860. The depicted illustrative embodiment of coupling article 820 also includes a bridge 882 connecting the leg 850 to leg 860 at a location distal from the anchor section 840 to which the capture section 880 is attached. The depicted embodiment of capture section 880 on coupling article 820 also includes a capture aperture 884. In the depicted illustrative embodiment of capture section 880 the capture aperture 884 may be described as being located between the anchor section 840, the bridge 882 and the legs 850 and 860.

The bridge 882 is, in one or more embodiments, configured for repositionable attachment to the carrier 823 providing the first major surface 821 of anchor section 840. In one or more embodiments in which the carrier 823 providing first major surface 821 of anchor section 840 is in the form of a mechanical fastener, the bridge 882 may include a mechanical fastener component 829 that is configured to attach to the mechanical fastener component provided on first major surface 821. Alternatively, the bridge 882 may be provided with an adhesive 829 that may be used to attach the bridge to the first major surface 821 of the anchor section 840. Also depicted in connection with the illustrative embodiment of coupling article 820 is a tab 886 at the distal end of the capture section 880, with the tab 886 facilitating manipulation of the capture section 882 capture a catheter as described herein.

The depicted illustrative embodiment of coupling article 820 also includes release liners 827 provided on legs 850 and 860 of capture section 880. In one or more embodiments, the release liners 827 may be provided to protect a pressure sensitive adhesive provided on the legs 850 and 860, with the adhesive on legs 850 and 860 being used to assist in capturing and securing a catheter using the coupling article 820.

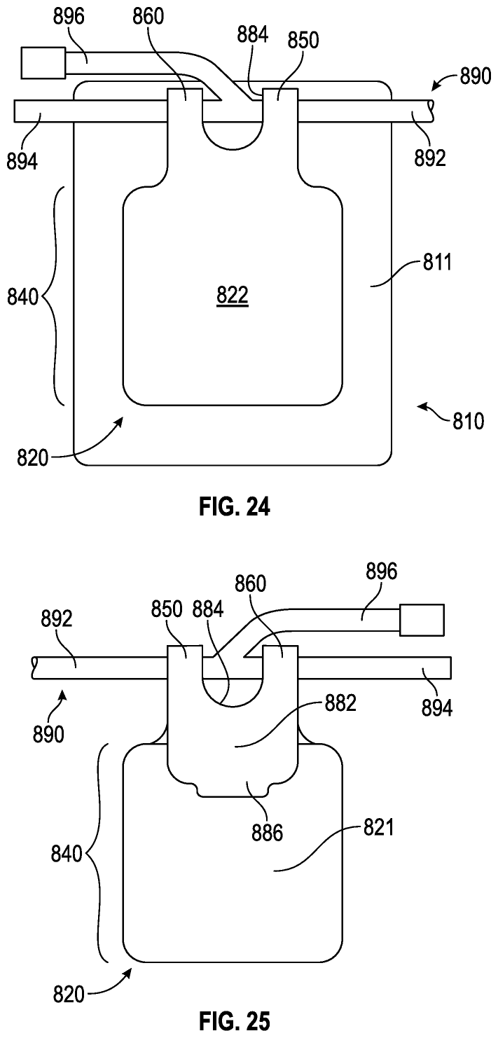

FIGS. 24-25 depict the coupling article 820 in use with a catheter 890 captured by the coupling article 820. The coupling article 820 is depicted as being attached to a base article 810 in FIG. 24 with the first major surface 821 of the anchor section 840 attached to the second major surface 811 of the base article 810 in FIG. 24. FIG. 25 depicts the coupling article 820 detached from the base article 810 of FIG. 24 such that the first major surface 821 of the anchor section 840 of the coupling article 820 is visible in FIG. 25.

The captured catheter 890 includes a supply tube 892 along with branch tubes 894 and 896 attached to the supply tube 892 at a Y-connection located in the capture aperture 884 between legs 850 and 860 of the capture section of the coupling article 820. Further, the bridge 882 of the capture section 880 is depicted as being attached to the first major surface 821 of the anchor section 840 when the catheter 890 is captured by the capture section 880 as depicted. When the coupling article 820 is depicted as attached to the base article 810 as seen in FIG. 24, at least a portion of the bridge 882 may be described as being located between the anchor section 840 and the base article 810 when the anchor section 840 is attached to the base article 810.

In one or more embodiments, the base articles 810 of catheter securement systems as depicted in FIGS. 21-25 may be described as having a base article footprint area while the anchor section 840 of the coupling article 820 may be described as having an anchor section footprint area. In one or more embodiments, the anchor section footprint area is smaller than the base article footprint area such that placement of the anchor section 840 on the base article may be facilitated. In one or more embodiments, the anchor section footprint area of the coupling articles may be 90% or less of the base article footprint area. In one or more alternative embodiments, the anchor section footprint area of the coupling articles may be 80% or less, 70% or less, or 60% or less of the base article footprint area. At the lower end of the range, it may be preferred that the anchor section may be large enough to occupy 10% or more, 20% or more, or 30% or more of the base article footprint area.

Regardless of the relative sizes of the base article footprint area and the anchor section footprint area of catheter securement systems as described herein, the size of the anchor section footprint areas of coupling articles described herein are preferably large enough to resist unwanted detachment of the coupling articles from base articles to which they are attached to secure a catheter at a selected location on a patient.

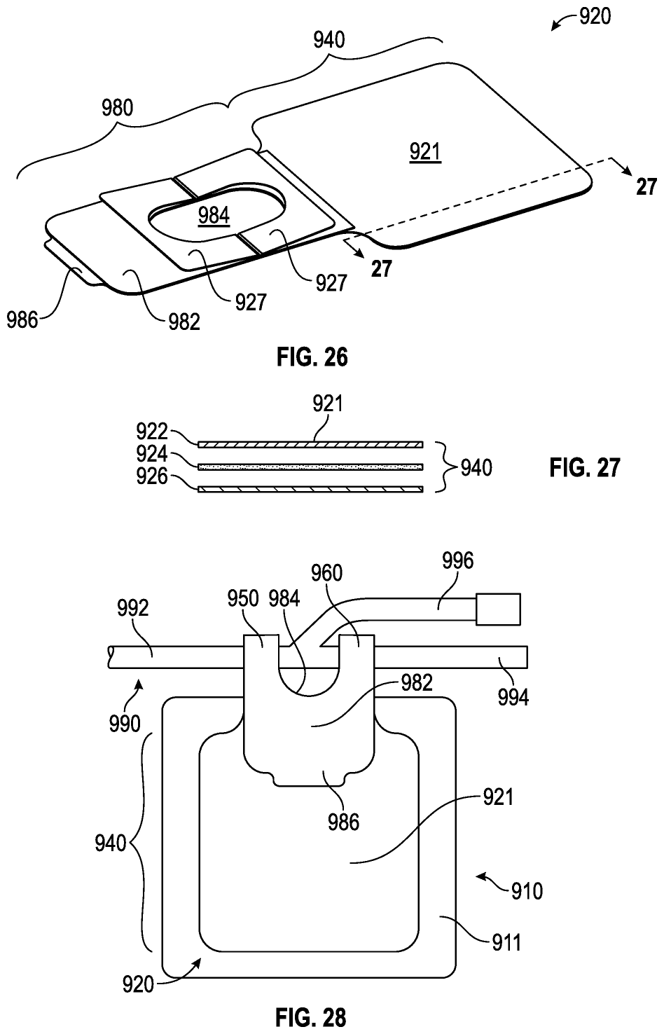

Yet another illustrative embodiment of a catheter securement system as described herein is depicted in connection with FIGS. 26-28. The catheter securement system depicted in those figures includes a coupling article 920 along with one or more base articles 910 to which the coupling articles may be repositionably attached as described herein.

Referring to FIGS. 26-28, the coupling article 920 includes an anchor section 940 and a capture section 980 attached to the anchor section 940. The anchor section 940 includes a first major surface 921 and a second major surface 922 on an opposite side of the anchor section 940 from the first major surface 921.

In one or more embodiments, the anchor section 940 may include multiple components with one illustrative embodiment of a set of components found in the depicted illustrative embodiment of anchor section 940 being depicted in the cross-sectional view of FIG. 27 which is taken along line 27-27 as seen in FIG. 26. The components found in the illustrative embodiment of anchor section 940 may include a backing layer 922 forming the first major surface 921 with adhesive 924 on the backing layer 922. The adhesive 922 is, in the depicted illustrative embodiment, protected by a release liner 926 which may be removed to allow attachment of the anchor section 942 a base article 910 as depicted in FIG. 28. In one or more alternative embodiments, adhesive 922 may be in the form of a non-tacky/cohesive polymeric material that is capable of securing the anchor section 940 of coupling article 922 a base article 910 as described herein.

In one or more alternative embodiments, the adhesive 924 may be replaced by a mechanical fastener component, with the base article 910 including a complementary mechanical fastener component such that the anchor section 940 of coupling article 920 can be repositionably attached to the base article 910 as described herein (e.g., in a manner similar to coupling article 220 depicted in FIGS. 9-13).

The depicted illustrative embodiment of coupling article 920 also includes a capture section 980 including legs 950 and 960. The depicted illustrative embodiment of coupling article 920 also includes a bridge 982 connecting the leg 950 to leg 960 at a location distal from the anchor section 940 to which the capture section 980 is attached. The depicted embodiment of capture section 980 on coupling article 920 also includes a capture aperture 984. In the depicted illustrative embodiment of capture section 980 the capture aperture 984 may be described as being located between the anchor section 940, the bridge 982 and the legs 950 and 960.

The bridge 982 is, in one or more embodiments, configured for repositionable attachment to the backing layer 922 providing the first major surface 921 of anchor section 940. The capture section 980, including bridge 982, may be provided with an adhesive that may be used to attach the bridge 982 to the first major surface 921 of the anchor section 940. Also depicted in connection with the illustrative embodiment of coupling article 920 is a tab 986 at the distal end of the capture section 980, with the tab 986 facilitating manipulation of the capture section 982 capture a catheter as described herein. The depicted illustrative embodiment of coupling article 920 also includes release liners 927 provided on capture section 980 to protect any adhesive located on the capture section 980 until used. In one or more embodiments, the release liners 927 may be provided to protect a pressure sensitive adhesive provided on the legs 950 and 960 as well as the bridge 982, with the adhesive on legs 950 and 960 being used to assist in capturing and securing a catheter using the coupling article 920.

In one or more alternative embodiments, the first major surface 921 of the anchor section 940 may include a mechanical fastener component and at least to the bridge 982 of the capture section 980 may include a complementary mechanical fastener component, with the two mechanical fastener components being used to secure the bridge to the anchor portion 940 and retain a catheter therein.

FIG. 28 depicts the coupling article 920 in use with a catheter 990 captured by the coupling article 920. The coupling article 920 is depicted as being attached to a base article 910 in FIG. 28 such that the first major surface 921 of the anchor section 940 faces away from the surface 911 of the base article 910 in FIG. 28.

The captured catheter 990 includes a supply tube 992 along with branch tubes 994 and 996 attached to the supply tube 992 at a Y-connection located in the capture aperture 984 between legs 950 and 960 of the capture section of the coupling article 920. Further, the bridge 982 of the capture section 980 is depicted as being attached to the first major surface 921 of the anchor section 940 when the catheter 990 is captured by the capture section 980 as depicted. When the coupling article 920 is depicted as attached to the base article 910 as seen in FIG. 28, at least a portion of the anchor section 940 may be described as being located between the bridge 982 and the base article 910 when the anchor section 940 is attached to the base article 910 and the capture section 980 is folded over to secure a catheter 990.

### Methods:

The catheter securement systems described herein to be used to secure a catheter to the skin of a patient. In one or more embodiments, two or more base articles may be provided to allow for movement of the catheter between the two or more base articles. In one or more embodiments, the catheter may be repositioned on the same base article using a catheter coupling as described herein.

In one or more embodiments, the methods of securing catheters using one or more embodiments of the catheter securement systems described herein may involve adhesively attaching a first base article to a first location on a patient using skin contact adhesive on the first base article. In addition, a second base article may be adhesively attached to a second location on a patient using skin contact adhesive on the second base article.

A coupling article of one or more embodiments of the catheter securement systems as described herein may be secured to a catheter such that the coupling article can be used to secure the catheter to the first base article. In one or more embodiments, the coupling article may be attached to the first base article before or after securing the coupling article to the catheter. In other words, the coupling article may first be attached to the catheter followed by attachment of the catheter and the coupling article to the first base article in one or more embodiments of the methods described herein. One example of such a method can be described in connection with FIG. 23, where the coupling article 820 may first be attached to the catheter 890, followed by attachment of both the coupling article 820 and the catheter 890 to a base article 810.

In one or more alternative embodiments, the coupling article may be attached to the base article before securing a catheter to the coupling article. One example of such a method can be described in connection with FIGS. 5-8, where the coupling article 120 may first be attached to the base article 110, followed by attachment of the catheter 192 the coupling article 120 and the base article 110.

The methods of securing catheters as described herein may involve removing a coupling article and catheter from a first base article followed by securing the catheter and coupling article to a second base article at a second location on a patient.

In one or more embodiments of methods of securing catheters as described herein, the method may further include removing a coupling article and catheter from a second base article to which they were moved followed by re-securing the catheter to the first base article using the same coupling article after removing that coupling article and the catheter from the second base article. In such methods, a user may repeatedly remove and re-secure the catheter and coupling article between two or more base articles as needed.

In one or more alternative embodiments of methods of securing catheters as described herein, the method may involve repositioning a catheter on the same base article by, e.g., removing the coupling article and the catheter from a first base article and re-securing the catheter to the first base article using the coupling article after removing the coupling article and the catheter from the first base article, with the catheter being potentially repositioned on the base article to which it is re-secured.

In one or more embodiments of methods as described herein, the coupling articles may remain secured to a catheter when moving a catheter between base articles or repositioning a catheter on the same base article. In one or more alternative embodiments of methods as described herein, the catheters may be removed from the coupling articles during movement of a catheter between base articles.

In one or more embodiments of methods as described herein in which the coupling article comprises a carrier, removing the coupling article from a base article may involve removing the carrier from the first base article, with the carrier then being attached to a second base article or repositioned on the same base article Although the catheters secured using various illustrative embodiments of the catheter securement systems as depicted in, e.g., FIGS. 5-8, 14-15, and 24-25 are bifurcated catheters, catheters secured using one or more embodiments of the catheter securement systems and/or methods described herein may or may not be bifurcated catheters. In other words, the catheter securement systems and/or methods described herein may be used to secure catheters having only one tube (and other articles including only one tube) where movement of the catheter (or other tube) between two different base articles on two different positions on a patient or repositioning on the same base article is desired as described herein.

### Adhesives:

As described herein, securing adhesives (e.g., adhesives 124 in FIG. 3, 224 in FIGS. 12, and 224′ in FIG. 15) used to attach the coupling articles to base articles of catheter securement systems or to other components in the coupling articles themselves (e.g., mechanical fastener material or another layer) as described herein can have an adhesion that is higher than the skin-contact adhesives used to adhesively attach the base articles to patient skin (e.g., the skin-contact adhesive 112 or 109 of FIG. 1). In some embodiments, the securing adhesive and the skin-contact adhesive may be of the same or similar classes of adhesive, but have different adhesion levels. For example, the securing adhesive and/or the skin-contact adhesive may be an acrylate, silicone, urethane, hydrogel, hydrocolloid, natural rubber, or synthetic rubber. Adhesion can also be tuned through changes in adhesive composition, adhesive thickness, or adhesive surface area (e.g., by employing a pattern-coated adhesive).

“Adhesion” refers to the force required to separate an adhesive from an underlying substrate. Adhesion can be measured in a number of ways. For example, adhesion can be defined by peel force or shear force. In some embodiments, adhesion can be defined by peel adhesion using ASTM D3330/D3330M-04 (2010). In some embodiments, adhesion can be defined by shear adhesion using ASTM D3654M-06 (2011). Adhesion is dependent on the specific substrate being adhered to, as well as the time the pressure-sensitive adhesive (PSA) is allowed to dwell on the substrate.

For example, typical peel adhesion values exhibited by pressure-sensitive adhesives in medical dressings maybe in the range of 20 to 300 g/cm as measured from stainless steel. In some embodiments, at least 10% higher peel adhesion, as measured by ASTM D3330/D3330M-04 (2010), of the securing adhesive over the skin-contact adhesive may realize the benefit of both securing to base article and, optionally a catheter, while providing gentle adhesion to the skin.

In some embodiments, the securing adhesive can be an acrylate adhesive and the skin-contact adhesive can be a silicone adhesive. The term “acrylate” or “acrylate-based” or “acrylate-containing” refers to monomeric acrylic or methacrylic esters of alcohols. Acrylate and methacrylate monomers are referred to collectively herein as “acrylate” monomers. Materials that are described as “acrylate-based” or “acrylate-containing” contain at least some acrylate monomers and may contain additional co-monomers.

Acrylate adhesives may be used for securing articles to each other (e.g., coupling articles to base articles and/or coupling articles to catheters) or, optionally, for securing the base articles to skin. The adhesion can be manipulated to have high adhesion or low adhesion. Generally, the adhesion between acrylate adhesives and another material will increase over time.

Examples of potentially suitable skin-contact pressure sensitive adhesives (PSAs) include rubber based adhesives (e.g., tackified natural rubbers, synthetic rubbers, and styrene block copolymers), acrylics (e.g., polymerized (meth)acrylates), poly(alpha-olefins), polyurethanes, and silicones. Amine containing polymers can also be used which have amine groups in the backbone, pendant thereof, or combinations thereof. A suitable example includes a poly(ethyleneimine).

Useful skin-contact adhesives can be any of those that are compatible with skin and useful for wound dressings, such as those disclosed in U.S. Pat. Nos. Re. 24,906 (Ulrich), U.S. Pat. No. 5,849,325 (Heinecke et al.), and U.S. Pat. No. 4,871,812 (Lucast et. al.) (water-based and solvent-based adhesives); U.S. Pat. No. 4,833,179 (Young et al.) (hot-melt adhesives); U.S. Pat. No. 5,908,693 (Delgado et al.) (microsphere adhesives); U.S. Pat. Nos. 6,171,985 and 6,083,856 (both to Joseph et al.) (low trauma fibrous adhesives); and, U.S. Pat. No. 6,198,016 (Lucast et al.), U.S. Pat. No. 6,518,343 (Lucast et al.), and U.S. Pat. No. 6,441,092 (Gieselman) (wet-skin adhesives). Inclusion of medicaments or antimicrobial agents in the adhesive is also contemplated, as described in U.S. Pat. No. 4,310,509 (Berglund) and U.S. Pat. No. 4,323,557 (Rosso).

Silicone and acrylic based pressure sensitive adhesives are commonly utilized for adhering to the skin.

Silicone PSAs typically include two major components, a polymer or gum, and a tackifying resin. The polymer is typically a high molecular weight polydimethylsiloxane or polydimethyl-diphenylsiloxane, that contains residual silanol functionality (SiOH) on the ends of the polymer chain, or a block copolymer including polydiorganosiloxane soft segments and urea terminated hard segments. The tackifying resin is generally a three-dimensional silicate structure that is endcapped with trimethylsiloxy groups (OSiMe₃) and also contains some residual silanol functionality. Examples of tackifying resins include SR 545, from General Electric Co., Silicone Resins Division, Waterford, N.Y., and MQD-32-2 from Shin-Etsu Silicones of America, Inc., Torrance, Calif. Manufacture of typical silicone PSAs is described in U.S. Pat. No. 2,736,721 (Dexter). Manufacture of silicone urea block copolymer PSA is described in U.S. Pat. No. 5,214,119 (Leir et al.). In some embodiments, the silicone adhesive may be characterized as gentle to skin such as described in U.S. Pat. No. 8,541,481 (Determan et al.), U.S. Patent Publication No. US2013/0040073 (Pett et al.), U.S. Pat. No. 7,407,709 (Zhou et al.) and U.S. Pat. No. 7,807,268 (Zhou et al.). Examples of suitable silicone adhesive systems can include, but are not limited to, products available under the following trade designations: Dow Corning MG 7-9850, Wacker SILPURAN® 2110 and 2130, Bluestar SILBIONE® RT Gel 4317 and 4320, Nusil MED-6345 and 6350.

Acrylic adhesive typically comprise a copolymer of at least one C₄-C₁₂ alkyl (meth)acrylate such as isooctyl acrylate or 2-ethylehexylacrylate and at least one high Tg (e.g. polar) comonomer such as (meth)acrylamide, N-vinyl pyrrolidone, poly(ethylene oxide)acrylate, and mixture thereof. In typical embodiments, the acrylic adhesive comprises at least 90 wt.-% C₄-C₁₂ alkyl (meth)acrylate(s). Suitable examples include a 90:10 isooctyl acrylate: acrylic acid copolymer, a 70:15:15 isooctyl acrylate: ethylene oxide acrylate: acrylic acid terpolymer, and a 25:69:6 2-ethylhexylacrylate: butyl acrylate: acrylic acid terpolymer. Another acrylic adhesive composition includes a 97:3 isooctyl acrylate: acrylamide copolymer 65:15:20 2-ethylhexylacrylate: acrylic acid: copolymer blended with a nonreactive polyalkylene oxide copolymer under the trade designation PLURONIC. Additional useful adhesives are described in U.S. Pat. No. 3,389,827 (Abere et al.), U.S. Pat. No. 4,112,213 (Waldman), U.S. Pat. No. 4,310,509 (Berglund et al.), and U.S. Pat. No. 4,323,557 (Rosso et al.).

For skin-contact adhesives, it is desirable that the adhesive is able to transmit moisture vapor at a rate greater to or equal to that of human skin. While such a characteristic can be achieved through the selection of an appropriate adhesive, it is also contemplated that other methods of achieving a high relative rate of moisture vapor transmission may be used, such as perforating the adhesive or pattern coating the adhesive, as described in U.S. Pat. No. 4,595,001 (Potter et al.) and U.S. Patent Application. Publication No. 2008-0233348 (also U.S. Pat. No. 7,947,366) (Ishiwatari et al.). Each of the securing or skin-contact adhesive can optionally be applied in a discontinuous manner.

### Backings:

Suitable backings for base articles and/or coupling articles of the catheter securement systems described herein can include, but are not limited to, one or more of a fabric, a woven fibrous web, a nonwoven fibrous web, a knit, a polymeric film, other familiar dressing materials, or combinations thereof. In some embodiments, the backing materials can include polymeric elastic films (e.g., transparent or non-transparent), and can include, but are not limited to, films formed of elastomeric polyurethanes, co-polyesters, polyethylenes, or combinations thereof. The backing can be a high moisture vapor permeable film, i.e., a backing with a relatively high moisture vapor transmission rate (MVTR). U.S. Pat. No. 3,645,835 (Hodgson) describes methods of making such films and methods for testing their permeability. The backing can be constituted of natural or synthetic sources of raw materials.

The backing layers of base articles and, optionally, coupling articles of one or more embodiments of catheter securement systems as described herein may preferably transmit moisture vapor at a rate equal to or greater than human skin. In some embodiments, the backing can be adhesive-coated. In such embodiments, the adhesive-coated backing can transmit moisture vapor at a rate of at least 300 g/m²/24 hrs/37° C./100-10% RH, and in some embodiments, at least 700 g/m²/24 hrs/37° C./100-10% RH. The backing is generally conformable to anatomical surfaces. As such, when the backing is applied to an anatomical surface, such as a nose, it conforms to the surface even when the surface is moved.

The backing layers can be a flexible material. For example, the backing layers can be a film, paper, woven, knit, foam, nonwoven material, or a combination thereof, or one or more layers of film, paper, woven, knit, foam, nonwoven, or a combination thereof. In some embodiments, it can be desirable that at least a portion of the backing layer is formed of a transparent material to allow for viewing of underlying skin, a medical device, and/or a target site.

By way of example only, in some embodiments, the backing of a base article as described herein can be formed of a film available under the trade designation TEGADERM™ from 3M Company, St. Paul, Minn.

In one or more embodiments, the backing layer of one or more embodiments of the base articles as described herein may be formed of a stretchable material (e.g., a stretchable nonwoven, woven, film, or combination thereof) that can provide gentle removal to reduce the potential for skin damage when the base articles are removed from a patient. For example, in one or more embodiments, the base article can include a stretch release backing (i.e., a backing formed of a stretch release material) and skin-contact adhesive, such that while stretching, there is a distribution of tension force between the backing, the adhesive, and the skin, providing adhesive failures and reducing the tension applied on the skin as the base article is removed.

By way of example only, in some embodiments, the backing layer of base articles and the skin-contact adhesive on them can be provided by polyurethane stretchable nonwoven tape, such as the tape available as 3M™ CoTran™ 9699 Melt Blow Polyurethane Tape from 3M Company, St. Paul, Minn., any of the materials A-L of Table 1 below, other suitable tapes/backings, or a combination thereof.

TABLE 1
Backing and Adhesive Combinations

| Material | Description | Source |
|---|---|---|
| A - Foam Tape | 3M ™ Polyethylene Foam Medical Tape 1774W, | 3M Company, |
| | 510 micron, closed cell, polyethylene foam | St. Paul, MN |
| | backing, coated with 60 micron thick pressure | |
| | sensitive acrylate adhesive. | |
| B - PU-NW Tape | 3M ™ CoTran ™ 9699 Melt Blown Polyurethane | 3M Company, |
| | Tape: 254 micron thick polyurethane/polyethylene | St. Paul, MN |
| | backing coated with a gentle medical acrylate | |
| | adhesive | |
| C - PU Film Tape | 3M ™ Polyurethane Tape 9834; 20, micron | 3M Company, |
| | polyurethane film with 25 micron thick gentle | St. Paul, MN |
| | medical acrylate adhesive | |
| D - coPET Film | 3M ™ Steri-Drape ™ 2 incise drape: | 3M Company, |
| Drape | 25 micron thick elastomeric copolyester | St. Paul, MN |
| | backing coated with 51 micron thick | |
| | pressure sensitive acrylate adhesive. | |
| E - Si Film | SILPURAN ™ Film 2030; medical grade silicone | Wacker Chemie AG, |
| | film, 100 micron thick | Munich Germany |
| F- PE Tape | 3M ™ Blenderm ™ Surgical Tape 1525 - | 3M Company, |
| | Polyethylene backing coated with gentle medical | St. Paul, MN |
| | acrylate adhesive | |
| G - LDPE Film | 3M ™ Steri-Drape ™ incise drape: 30 micron thick | 3M Company, |
| Drape | low density polyethylene backing coated with 51 | St. Paul, MN |
| | micron thick pressure sensitive acrylate adhesive | |
| H - coPET-AM | 3M ™ Ioban ™ 2 Antimicrobial incise Drape; 25 | 3M Company, |
| Drape | micron thick elastomeric copolyester backing | St. Paul, MN |
| | coated with 51 micron thick iodophor impregnated | |
| | (antimicrobial) pressure sensitive acrylate adhesive | |
| I - Nonwoven | 3M ™ Micropore ™ Surgical Tape 1530 - Rayon | 3M Company, |
| Backing Tape | backing coated with medical acrylate adhesive | St. Paul, MN |
| J - PET Nonwoven | 3M ™ Medipore ™ H Surgical Tape (2864) - | 3M Company, |
| Tape | Polyester nonwoven backing coated with medical | St. Paul, MN |
| | acrylate adhesive | |
| K- Hydrocolloid | 3M ™ Polyurethane backing with hydrocolloid | 3M Company, |
| Tape | medical adhesive (9943) | St. Paul, MN |
| L - Cloth Tape | 3M ™ Durapore ™ Surgical Tape (1538) - a silk- | 3M Company, |
| | like fabric backing with adhesive | St. Paul, MN |

### Release Liners:

Release liners suitable for use with the catheter securement systems described herein can include, but are not limited to, kraft papers, polyethylene, embossed polyethylene, polypropylene, polyester, or combinations thereof. Such liners can be coated with release agents, such as fluorochemicals, silicones, or other suitable low surface energy materials. Other adhesives and release liner combinations known to those of ordinary skill in the art can also be employed in the catheter securement systems described herein. Examples of commercially available silicone coated release papers are POLYSLIK™, silicone release papers available from Rexam Release (Bedford Park, Ill.) and silicone release papers supplied by LOPAREX (Willowbrook, Ill.). Other non-limiting examples of such release liners commercially available include siliconized polyethylene terephthalate films, commercially available from H. P. Smith Co., and fluoropolymer coated polyester films, commercially available from 3M Company (St. Paul) under the brand “SCOTCHPAK™” release liners.

Each embodiment shown in the figures is illustrated as a separate embodiment for clarity in illustrating a variety of features of the systems, kits, or methods described herein. However, it should be understood that any combination of elements and features of any of the embodiments illustrated in the figures and described herein can be employed in the systems, kits, or methods described herein.

### EMBODIMENTS

The following embodiments are intended to be illustrative and not limiting.

1. A catheter securement system, the system comprising:

a base article configured to be adhered to skin, the base article comprising a first major surface comprising a skin-contact adhesive and a second major surface opposite the first major surface; and

a coupling article comprising:
a carrier comprising a first major surface configured for repositionable attachment to the second major surface of the base article and a second major surface facing away from the first major surface;
an anchor section attached to the second major surface of the carrier; and
a capture section attached to the anchor section, the capture section comprising a first leg extending away from the anchor section, the first leg comprising a first end located distal from the anchor section.
   2. A system according to embodiment 1, wherein the first end of the first leg is configured for repositionable attachment to the carrier.
   3. A system according to any one of embodiments 1 to 2, wherein the capture section of the coupling article comprises a second leg extending away from the anchor section, wherein the second leg is adjacent the first leg, and wherein the second leg comprises a second end located distal from the anchor section.
   4. A system according to embodiment 3, wherein the second end of the second leg is configured for repositionable attachment to the carrier.
   5. A system according to any one of embodiments 3 to 4, wherein the first leg extends from the anchor section to the first end along a first direction, wherein the second leg extends from the anchor section to the second end along a second direction, and wherein the first direction and the second direction are aligned with each other.
   6. A system according to any one of embodiments 3 to 5, wherein the capture section comprises a bridge connecting the first leg to the second leg at a location distal from the anchor section, wherein the bridge is configured for repositionable attachment to the carrier.
   7. A system according to embodiment 6, wherein the capture section comprises a capture aperture located between the anchor section, the bridge, the first leg, and the second leg.
   8. A system according to any one of embodiments 1 to 2, wherein the anchor section comprises a first anchor section and wherein the capture section comprise a first capture section, and wherein the coupling article further comprises a second anchor section attached to the second major surface of the carrier and a second capture section attached to the second anchor section, the second capture section comprising a second leg extending away from the second anchor section, the second leg comprising a second end located distal from the second anchor section.
   9. A system according to embodiment 8, wherein the second anchor section is located adjacent the first anchor section.
   10. A system according to embodiment 8, wherein the first anchor section is located proximate the second end of the second leg, and wherein the second anchor section is located proximate the first end of the first leg.
   11. A system according to any one of embodiments 1 to 10, wherein the coupling article comprises a first mechanical fastener component attached to the first major surface of the carrier, and wherein a second mechanical fastener component is attached to the second major surface of the base article, wherein the coupling article is configured for repositionable attachment to the base article through engagement of the first mechanical fastener component with the second mechanical fastener component.
   12. A system according to any one of embodiments 1 to 10, wherein the carrier comprises a coupling article layer of non-tacky/cohesive polymeric material on the first major surface of the coupling article, wherein the coupling article is configured for attachment to the base article using the non-tacky/cohesive polymeric material on the first major surface of the coupling article.
   13. A system according to embodiment 12, wherein the base article comprises a base article layer of non-tacky/cohesive polymeric material on the second major surface of the base article, wherein the coupling article layer and the base article layer of the non-tacky/cohesive polymeric material provide repositionable attachment of the coupling article to the base article.
   14. A system according to any one of embodiments 1 to 10, wherein the coupling article comprises carrier pressure sensitive adhesive on the first major surface of the carrier, wherein the coupling article is configured for repositionable adhesive attachment to the base article using the carrier pressure sensitive adhesive.
   15. A system according to any one of embodiments 1 to 14, wherein the capture section of the coupling article comprise securing pressure sensitive adhesive configured to attach to the carrier.
   16. A system according to embodiment 15, wherein the coupling article comprises a capture section release liner releasably attached to the capture pressure sensitive adhesive.
   17. A system according to any one of embodiments 1-11, wherein the capture section comprises a third mechanical fastener component attached to the capture section, wherein the capture section is configured for repositionable attachment to the carrier through engagement of the third mechanical fastener component on the capture section with the first mechanical fastener component on the carrier.
   18. A system according to any one of embodiments 1 to 17, wherein the base article comprises a base article footprint area, and wherein the carrier of the coupling article comprises a coupling article footprint area that is smaller than the base article footprint area.
   19. A system according to embodiment 18, wherein the coupling article footprint area is 90% or less, 80% or less, 70% or less, or 60% or less of the base article footprint area. At the lower end of the range, the coupling article footprint area may be 10% or more, 20% or more or 30% or more of the base article footprint area.
   20. A system according to any one of embodiments 1 to 19, wherein the base article comprises a base article backing that comprises a stretch release material.
   21. A system according to any one of embodiments 1 to 20, wherein the base article comprises a first base article, and wherein the system further comprises a second base article configured to be adhered to skin, the second base article comprising a first major surface comprising a skin-contact adhesive and a second major surface opposite the first major surface;

and wherein the carrier of the coupling article is configured for repositionable attachment to the second major surface of the second base article.

22. A catheter securement system, the system comprising:

a base article configured to be adhered to skin, the base article comprising a first major surface comprising a skin-contact adhesive and a second major surface opposite the first major surface; and

a coupling article configured for repositionable attachment to the second major surface of the base article, the coupling article comprising:
an anchor section;
a capture section attached to the anchor section, the capture section comprising:
   a first leg extending away from the anchor section, the first leg comprising a first end located distal from the anchor section;
   a second leg extending away from the anchor section, the second leg comprising a second end located distal from the anchor section;
   a bridge connecting the first leg to the second leg at a location distal from the anchor section, wherein the bridge is configured for repositionable attachment to the anchor section; and
   a capture aperture located between the anchor section, the bridge, the first leg, and the second leg.
      23. A system according to embodiment 22, wherein the bridge is configured for attachment to the anchor section.
      24. A system according to embodiment 23, wherein the bridge is configured for repositionable attachment to the anchor section.
      25. A system according to any one of embodiments 22 to 24, wherein the coupling article comprises a first mechanical fastener component attached to the anchor section, and wherein a second mechanical fastener component is attached to the second major surface of the base article, wherein the coupling article is configured for repositionable attachment to the base article through engagement of the first mechanical fastener component with the second mechanical fastener component.
      26. A system according to embodiment 25, wherein the first mechanical fastener component is located between the anchor section and the base article when the coupling article is attached to the base article.
      27. A system according to any one of embodiments 25 to 26, wherein the capture section comprises a third mechanical fastener component attached to the bridge, wherein the capture section is configured for repositionable attachment to the anchor section through engagement of the third mechanical fastener component on the bridge with the first mechanical fastener component on the anchor section.
      28. A system according to any one of embodiments 22 to 24, wherein the coupling article comprises pressure sensitive adhesive on the anchor section, wherein the anchor section of the coupling article is configured for repositionable adhesive attachment to the base article using the pressure sensitive adhesive.
      29. A system according to any one of embodiments 22 to 28, wherein the bridge of the capture section comprises pressure sensitive adhesive configured to adhesively attach the bridge to the anchor section.
      30. A system according to embodiment 29, wherein the coupling article comprises a capture section release liner releasably attached to the pressure sensitive adhesive on the bridge.
      31. A system according to any one of embodiments 22 to 30, wherein at least a portion of the bridge is located between the anchor section and the base article when the anchor section is attached to the base article.
      32. A system according to any one of embodiments 22 to 30, wherein at least a portion of the anchor section is located between the bridge and the base article when a catheter is captured in the capture section.
      33. A system according to any one of embodiments 22 to 32, wherein the base article comprises a base article footprint area, and wherein the anchor section of the coupling article comprises an anchor section footprint area that is smaller than the base article footprint area.
      34. A system according to embodiment 33, wherein the anchor section footprint area is 90% or less of the base article footprint area.
      35. A system according to any one of embodiments 22 to 34, wherein the base article comprises a first base article, and wherein the system further comprises a second base article configured to be adhered to skin, the second base article comprising a first major surface comprising a skin-contact adhesive and a second major surface opposite the first major surface;
and wherein the coupling article is configured for repositionable attachment to the second major surface of the second base article.
   36. A catheter securement system, the system comprising:

a base article configured to be adhered to skin, the base article comprising a first major surface comprising a skin-contact adhesive and a second major surface opposite the first major surface; and

a coupling article comprising a first major surface configured for repositionable attachment to the second major surface of the base article, the coupling article comprising;
an anchor section; and
a capture section attached to the anchor section, the capture section comprising a first leg extending away from the anchor section, the first leg comprising a first end located distal from the anchor section.
   37. A system according to embodiment 36, wherein the first end of the first leg is configured for repositionable attachment to the base article.
   38. A system according to any one of embodiments 36 to 37, wherein the capture section of the coupling article comprises a second leg extending away from the anchor section, wherein the second leg is adjacent the first leg, and wherein the second leg comprises a second end located distal from the anchor section.
   39. A system according to embodiment 38, wherein the second end of the second leg is configured for repositionable attachment to the base article.
   40. A system according to any one of embodiments 38 to 39, wherein the first leg extends from the anchor section to the first end along a first direction, wherein the second leg extends from the anchor section to the second end along a second direction, and wherein the first direction and the second direction are aligned with each other.
   41. A system according to any one of embodiments 38 to 40, wherein the capture section comprises a bridge connecting the first leg to the second leg at a location distal from the anchor section, wherein the bridge is configured for repositionable attachment to the base article.
   42. A system according to embodiment 41, wherein the capture section comprises a capture aperture located between the anchor section, the bridge, the first leg, and the second leg.
   43. A system according to any one of embodiments 36 to 37, wherein the anchor section comprises a first anchor section and wherein the capture section comprise a first capture section, and wherein the coupling article further comprises a second anchor section and a second capture section attached to the second anchor section, the second capture section comprising a second leg extending away from the second anchor section, the second leg comprising a second end located distal from the second anchor section.
   44. A system according to embodiment 43, wherein the second anchor section is located adjacent the first anchor section.
   45. A system according to embodiment 43, wherein the first anchor section is located proximate the second end of the second leg, and wherein the second anchor section is located proximate the first end of the first leg.
   46. A system according to any one of embodiments 36 to 45, wherein the coupling article comprises a first mechanical fastener component attached to the anchor section, and wherein a second mechanical fastener component is attached to the second major surface of the base article, wherein the coupling article is configured for repositionable attachment to the base article through engagement of the first mechanical fastener component with the second mechanical fastener component.
   47. A system according to any one of embodiments 36 to 45, wherein the coupling article comprises a coupling article layer of non-tacky/cohesive polymeric material on the first major surface of the coupling article, wherein the coupling article is configured for repositionable attachment to the base article using the non-tacky/cohesive polymeric material on the first major surface of the coupling article.
   48. A system according to embodiment 47, wherein the base article comprises a base article layer of non-tacky/cohesive polymeric material on the second major surface of the base article, wherein the coupling article layer and the base article layer of the non-tacky/cohesive polymeric material provide repositionable attachment of the coupling article to the base article.
   49. A system according to any one of embodiments 36 to 45, wherein the coupling article comprises pressure sensitive adhesive on the anchor section, wherein the coupling article is configured for repositionable adhesive attachment to the base article using the pressure sensitive adhesive on the anchor section.
   50. A system according to embodiment 49, wherein the coupling article comprises an anchor section release liner releasably attached to the pressure sensitive adhesive on the anchor section.
   51. A system according to any one of embodiments 36 to 50, wherein the capture section of the coupling article comprises capture pressure sensitive adhesive configured to attach to the base article.
   52. A system according to embodiment 51, wherein the coupling article comprises a capture section release liner releasably attached to the capture pressure sensitive adhesive.
   53. A system according to any one of embodiments 36 to 52, wherein the base article comprises a base article footprint area, and wherein the coupling article comprises a coupling article footprint area that is smaller than the base article footprint area.
   54. A system according to embodiment 53, wherein the coupling article footprint area is 90% or less of the base article footprint area.
   55. A system according to any one of embodiments 36 to 54, wherein the base article comprises a first base article, and wherein the system further comprises a second base article configured to be adhered to skin, the second base article comprising a first major surface comprising a skin-contact adhesive and a second major surface opposite the first major surface;

and wherein the coupling article is configured for repositionable attachment to the second major surface of the second base article.

56. A system according to any one of embodiments 1 to 55, wherein the base article comprises a base article backing that comprises a stretch release material.
57. A kit comprising a package containing the catheter securement system of any one of the preceding embodiments, wherein the base article of the catheter securement system comprises a first base article, and wherein the package contains a second base article configured to be adhered to skin, the second base article comprising a first major surface comprising a skin-contact adhesive and a second major surface opposite the first major surface.
58. A kit according to embodiment 57, wherein the first base article and the second base article are attached to a kit sheet located in the package.
59. A kit according to embodiment 58, wherein the coupling article is attached to the kit sheet.
60. A kit according to any one of embodiments 57 to 58, wherein the coupling article is attached to the second major surface of the first base article in the package.
61. A kit according to any one of embodiments 58 to 60, wherein the kit sheet comprises a first major surface comprising a release surface, and wherein the first base article and the second base article are adhesively attached to the release surface of the kit sheet.
62. A method of securing a catheter, the method comprising:
adhesively attaching a first base article to a first location on a patient using skin-contact adhesive on a first major surface of the first base article;
adhesively attaching a second base article to a second location on a patient using skin-contact adhesive on a first major surface of the second base article;
securing a coupling article to a catheter;
securing the catheter to the first base article using a coupling article;
removing the coupling article and the catheter from the first base article; and
securing the catheter to the second base article using the coupling article after removing the coupling article and the catheter from the first base article.
   63. A method according to embodiment 62, wherein the method further comprises:
removing the coupling article and the catheter from the second base article; and
re-securing the catheter to the first base article using the coupling article after removing the coupling article and the catheter from the second base article.
   64. A method according to any one of embodiments 62 to 63, wherein the coupling article remains secured to the catheter when removing the coupling article and the catheter from the first base article, and when securing the catheter to the second base article using the coupling article after removing the coupling article and the catheter from the first base article.
   65. A method according to any one of embodiments 62 to 64, wherein securing the coupling article to the catheter occurs before securing the catheter to the first base article.
   66. A method according to any one of embodiments 62 to 64, wherein the coupling article is attached to the first base article before securing the coupling article to the catheter such that securing the catheter to the first base article occurs at the same time as securing the coupling article to the catheter.
   67. A method according to any one of embodiments 62 to 66, wherein securing the catheter to the second base article comprises adhesively attaching the coupling article to the second base article.
   68. A method according to any one of embodiments 62 to 66, wherein securing the catheter to the second base article comprises attaching the coupling article to the second base article using a mechanical fastener component.
   69. A method according to any one of embodiments 62 to 66, wherein securing the catheter to the second base article comprises attaching the coupling article to the second base article using a non-tacky/cohesive polymeric material.
   70. A method according to any one of embodiments 62 to 69, wherein the catheter comprises a supply tube, a first branch tube, and a second branch tube, wherein the supply tube, the first branch tube, and the second branch tube meet at the Y-junction and wherein the coupling article captures the supply tube on one side of the Y-junction and the first branch tube on the opposite side of the Y-junction.
   71. A method according to embodiment 70, wherein the supply tube and the first branch tube remain captured by the coupling article when on the opposite side of the Y-junction when removing the coupling article and the catheter from the first base article and securing the catheter to the second base article using the coupling article after removing the coupling article and the catheter from the first base article.
   72. A method according to any one of embodiments 62 to 71, wherein the coupling article comprises a carrier, and wherein removing the coupling article and the catheter from the first base article comprises removing the carrier from the first base article, and further wherein securing the catheter to the second base article using the coupling article after removing the coupling article and the catheter from the first base article comprises attaching the carrier to the second base article.
   73. A method of securing a catheter, the method comprising:
adhesively attaching a first base article to a first location on a patient using skin-contact adhesive on a first major surface of the first base article;
securing a coupling article to a catheter;
securing the catheter to the first base article using a coupling article;
removing the coupling article and the catheter from the first base article; and
re-securing the catheter to the first base article using the coupling article after removing the coupling article and the catheter from the first base article.
   74. A method according to embodiment 73, wherein the coupling article remains secured to the catheter when removing the coupling article and the catheter from the first base article, and when re-securing the catheter to the first base article using the coupling article after removing the coupling article and the catheter from the first base article.
   75. A method according to any one of embodiments 73 to 74, wherein securing the coupling article to the catheter occurs before securing the catheter to the first base article.
   76. A method according to any one of embodiments 73 to 74, wherein the coupling article is attached to the first base article before securing the coupling article to the catheter such that securing the catheter to the first base article occurs at the same time as securing the coupling article to the catheter.
   77. A method according to any one of embodiments 73 to 76, wherein securing the catheter to the first base article comprises adhesively attaching the coupling article to the first base article.
   78. A method according to any one of embodiments 73 to 76, wherein securing the catheter to the first base article comprises attaching the coupling article to the first base article using a mechanical fastener component.
   79. A method according to any one of embodiments 73 to 76, wherein securing the catheter to the first base article comprises attaching the coupling article to the first base article using a non-tacky/cohesive polymeric material.
   80. A method according to any one of embodiments 73 to 79, wherein the catheter comprises a supply tube, a first branch tube, and a second branch tube, wherein the supply tube, the first branch tube, and the second branch tube meet at the Y-junction and wherein the coupling article captures the supply tube on one side of the Y-junction and the first branch tube on the opposite side of the Y-junction.
   81. A method according to embodiment 80, wherein the supply tube and the first branch tube remain captured by the coupling article when on the opposite side of the Y-junction when removing the coupling article and the catheter from the first base article and re-securing the catheter to the first base article using the coupling article after removing the coupling article and the catheter from the first base article.
   82. A method according to any one of embodiments 73 to 81, wherein the coupling article comprises a carrier, and wherein removing the coupling article and the catheter from the first base article comprises removing the carrier from the first base article, and further wherein re-securing the catheter to the first base article using the coupling article after removing the coupling article and the catheter from the first base article comprises attaching the carrier to the first base article.
   83. A method according to any one of embodiments 61 to 82, wherein the coupling article and the first base article comprise the catheter securement system of any one of embodiments 1 to 56.
   84. A method of securing a catheter, the method comprising:
adhesively attaching the base article of any one of embodiments 1 to 56 to a first location on a patient using skin-contact adhesive on a first major surface of the base article;
securing a coupling article according to any one of embodiments 1 to 56 to a catheter; and
   securing the catheter to the base article using the coupling article.

The complete disclosure of the patents, patent documents, and publications identified herein are incorporated by reference in their entirety as if each were individually incorporated. To the extent there is a conflict or discrepancy between this document and the disclosure in any such incorporated document, this document will control.

From the above disclosure of the general principles of the present invention and the preceding detailed description, those skilled in this art will readily comprehend the various modifications, re-arrangements and substitutions to which the present invention is susceptible, as well as the various advantages and benefits the present invention may provide. Therefore, the scope of the invention should be limited only by the following claims and equivalents thereof. In addition, it is understood to be within the scope of the present invention that the disclosed and claimed systems, kits and methods may be useful in other applications. Therefore, the scope of the invention may be broadened to include the use of the claimed and disclosed methods for such other applications.

1. A catheter securement system, the system comprising:
a base article configured to be adhered to skin, the base article comprising a first major surface comprising a skin-contact adhesive and a second major surface opposite the first major surface; and
a coupling article comprising:
a carrier comprising a first major surface configured for repositionable attachment to the second major surface of the base article and a second major surface facing away from the first major surface;
an anchor section attached to the second major surface of the carrier; and
a capture section attached to the anchor section, the capture section comprising a first leg extending away from the anchor section, the first leg comprising a first end located distal from the anchor section.

2. A system according to claim 1, wherein the first end of the first leg is configured for repositionable attachment to the carrier.

3. A system according to claim 1, wherein the capture section of the coupling article comprises a second leg extending away from the anchor section, wherein the second leg is adjacent the first leg, and wherein the second leg comprises a second end located distal from the anchor section.

4. A system according to claim 3, wherein the second end of the second leg is configured for repositionable attachment to the carrier.

5. A system according to claim 3, wherein the first leg extends from the anchor section to the first end along a first direction, wherein the second leg extends from the anchor section to the second end along a second direction, and wherein the first direction and the second direction are aligned with each other.

6. A system according to claim 3, wherein the capture section comprises a bridge connecting the first leg to the second leg at a location distal from the anchor section, wherein the bridge is configured for repositionable attachment to the carrier.

7. A system according to claim 6, wherein the capture section comprises a capture aperture located between the anchor section, the bridge, the first leg, and the second leg.

8. A system according to claim 1, wherein the anchor section comprises a first anchor section and wherein the capture section comprise a first capture section, and wherein the coupling article further comprises a second anchor section attached to the second major surface of the carrier and a second capture section attached to the second anchor section, the second capture section comprising a second leg extending away from the second anchor section, the second leg comprising a second end located distal from the second anchor section.

9. A system according to claim 8, wherein the second anchor section is located adjacent the first anchor section.

10. A system according to claim 8, wherein the first anchor section is located proximate the second end of the second leg, and wherein the second anchor section is located proximate the first end of the first leg.

11. A system according to claim 1, wherein the coupling article comprises a first mechanical fastener component attached to the first major surface of the carrier, and wherein a second mechanical fastener component is attached to the second major surface of the base article, wherein the coupling article is configured for repositionable attachment to the base article through engagement of the first mechanical fastener component with the second mechanical fastener component.

12.-21. (canceled)

22. A catheter securement system, the system comprising:
a base article configured to be adhered to skin, the base article comprising a first major surface comprising a skin-contact adhesive and a second major surface opposite the first major surface; and
a coupling article configured for repositionable attachment to the second major surface of the base article, the coupling article comprising:
an anchor section;
a capture section attached to the anchor section, the capture section comprising:
a first leg extending away from the anchor section, the first leg comprising a first end located distal from the anchor section;
a second leg extending away from the anchor section, the second leg comprising a second end located distal from the anchor section;
a bridge connecting the first leg to the second leg at a location distal from the anchor section, wherein the bridge is configured for repositionable attachment to the anchor section; and
a capture aperture located between the anchor section, the bridge, the first leg, and the second leg.

23. A system according to claim 22, wherein the bridge is configured for attachment to the anchor section.

24. A system according to claim 23, wherein the bridge is configured for repositionable attachment to the anchor section.

25. A system according to claim 22, wherein the coupling article comprises a first mechanical fastener component attached to the anchor section, and wherein a second mechanical fastener component is attached to the second major surface of the base article, wherein the coupling article is configured for repositionable attachment to the base article through engagement of the first mechanical fastener component with the second mechanical fastener component.

26. A system according to claim 25, wherein the first mechanical fastener component is located between the anchor section and the base article when the coupling article is attached to the base article.

27. (canceled)

28. A system according to claim 22, wherein the coupling article comprises pressure sensitive adhesive on the anchor section, wherein the anchor section of the coupling article is configured for repositionable adhesive attachment to the base article using the pressure sensitive adhesive.

29.-61. (canceled)

62. A method of securing a catheter, the method comprising:
adhesively attaching a first base article to a first location on a patient using skin-contact adhesive on a first major surface of the first base article;
adhesively attaching a second base article to a second location on a patient using skin-contact adhesive on a first major surface of the second base article;
securing a coupling article to a catheter;
securing the catheter to the first base article using a coupling article;
removing the coupling article and the catheter from the first base article; and
securing the catheter to the second base article using the coupling article after removing the coupling article and the catheter from the first base article.

63. A method according to claim 62, wherein the method further comprises:
removing the coupling article and the catheter from the second base article; and
re-securing the catheter to the first base article using the coupling article after removing the coupling article and the catheter from the second base article.

64. A method according to claim 62, wherein the coupling article remains secured to the catheter when removing the coupling article and the catheter from the first base article, and when securing the catheter to the second base article using the coupling article after removing the coupling article and the catheter from the first base article.

65.-84. (canceled)
